SEQUENCE LISTING (20 sequences)

SEQ ID NO: 1 - RNA, 19 nt, synthetic construct
  source 1..19: mol_type = other RNA
ggcttgatcatgaactact

SEQ ID NO: 2 - RNA, 21 nt, synthetic construct
  source 1..21: mol_type = other RNA
agtagttcatgatcaagccag

SEQ ID NO: 3 - RNA, 19 nt, synthetic construct
  source 1..19: mol_type = other RNA
gcagctccttattgttata

SEQ ID NO: 4 - RNA, 21 nt, synthetic construct
  source 1..21: mol_type = other RNA
tataacaataaggagctgcca

SEQ ID NO: 5 - RNA, 19 nt, synthetic construct
  source 1..19: mol_type = other RNA
gctccttattgttatacga

SEQ ID NO: 6 - RNA, 21 nt, synthetic construct
  source 1..21: mol_type = other RNA
tcgtataacaataaggagctg

SEQ ID NO: 7 - RNA, 19 nt, synthetic construct
  source 1..19: mol_type = other RNA
cggtaatggacagagttat

SEQ ID NO: 8 - RNA, 21 nt, synthetic construct
  source 1..21: mol_type = other RNA
ataactctgtccattaccgtg

SEQ ID NO: 9 - RNA, 19 nt, synthetic construct
  source 1..19: mol_type = other RNA
ctggcttgatcaagaacta

SEQ ID NO: 10 - RNA, 21 nt, synthetic construct
  source 1..21: mol_type = other RNA
tagttcttgatcaagccagca

SEQ ID NO: 11 - RNA, 19 nt, synthetic construct
  source 1..19: mol_type = other RNA
agaggacaacagaatatta

SEQ ID NO: 12 - RNA, 21 nt, synthetic construct
  source 1..21: mol_type = other RNA
taatattctgttgtcctctga

SEQ ID NO: 13 - RNA, 19 nt, synthetic construct
  source 1..19: mol_type = other RNA
ctgacacaatgctcagaaa

SEQ ID NO: 14 - RNA, 21 nt, synthetic construct
  source 1..21: mol_type = other RNA
tttctgagcattgtgtcagat

SEQ ID NO: 15 - DNA, 18 bp, synthetic construct
  source 1..18: mol_type = other DNA
gacacgatgctcagatgc

SEQ ID NO: 16 - DNA, 18 bp, synthetic construct
  source 1..18: mol_type = other DNA
ggggtttcctcagtcagt

SEQ ID NO: 17 - DNA, 17 bp, synthetic construct
  source 1..17: mol_type = other DNA
cgtccctccgaatgtta

SEQ ID NO: 18 - DNA, 20 bp, synthetic construct
  source 1..20: mol_type = other DNA
cgttggctacccgtgatatt

SEQ ID NO: 19 - DNA, 22 bp, synthetic construct
  source 1..22: mol_type = other DNA
ctcgtcaagaaggcgatagaag

SEQ ID NO: 20 - DNA, 24 bp, synthetic construct
  source 1..24: mol_type = other DNA
ccgcttcctcgtgctttacggtat

(19) United States
(12) Patent Application Publication
(10) Pub. No.: US 2026/0009029 A1
(43) Pub. Date: Jan. 8, 2026

(54) COMPOUND FOR INHIBITING EXPRESSION OF LPA GENE, PHARMACEUTICAL COMPOSITION AND USE THEREOF

(71) Applicant: RIGERNA THERAPEUTICS (SUZHOU) CO., LTD., SUZHOU, JIANGSU (CN)

(72) Inventors: YUANYU HUANG, SUZHOU, JIANGSU (CN); HAITAO LI, SUZHOU, JIANGSU (CN); LINA KONG, SUZHOU, JIANGSU (CN); XIAOFENG HAN, SUZHOU, JIANGSU (CN)

(21) Appl. No.: 19/127,433

(22) PCT Filed: Apr. 9, 2024
(86) PCT No.: PCT/CN2024/086781

§ 371 Date: May 6, 2025

(30) Foreign Application Priority Data
Apr. 14, 2023 (WO) PCT/CN2023/088483
Aug. 22, 2023 (CN) 202311061772.1

(51) Int. Cl. C12N 15/113

(52) U.S. Cl. CPC C12N 15/113; C12N 2310/14; C12N 2310/351

(57) ABSTRACT

Provided in the present disclosure are a compound for inhibiting the expression of the LPA gene, a pharmaceutical composition and the use thereof, which belong to the technical field of small nucleic acid drug delivery. The oligonucleotide-conjugated compound and the pharmaceutical composition thereof provided in the present disclosure can significantly inhibit the expression of LPA mRNA in animal liver tissues. The oligonucleotide-conjugated compound and the pharmaceutical composition thereof provided in the present disclosure are helpful for relieving, preventing and/or treating diseases or conditions mediated by LPA gene expression dysregulation.
AA Relative expression level of LPA mRNA (%)
BB Level of LPA mRNA in liver tissues of Balb/c-HDI mice
CC s.c. single dose, 1 mg/kg

### FIELD OF THE DISCLOSURE

The present disclosure relates to the technical field of delivery of small nucleic acid drugs, and particularly relates to a compound conjugated with an oligonucleotide for use in inhibiting expression of LPA gene, a pharmaceutical composition, and use thereof.

### BACKGROUND OF THE DISCLOSURE

Lipoprotein(a) (Lp(a), LPA) is a low-density lipoprotein variant consisting of a covalent combination of the Apo(a) isoform of the high molecular weight glycoprotein homologous to plasminogen (PLG) and LDL-like particles forming a disulfide bond, and is a low-density lipoprotein-like protein with an apo(a) motif. LPA is the name of the gene encoding apolipoprotein(a) (apo(a)). It is expressed predominantly in the liver and its expression is limited to humans and non-primates. Plasma Lp(a) concentrations are mostly determined by the LPA gene, which plays a key role in revealing the causal relationship between Lp(a) and cardiovascular disease. Lp(a) levels are a direct causative factor in a variety of cardiovascular diseases and are closely associated with cardiovascular diseases such as myocardial infarction, lower extremity arteriopathy, peripheral atherosclerosis, and aortic stenosis. Using Mendelian randomization studies, elevated Lp(a) levels have been confirmed to be a cause of cardiovascular disease, Lp(a) has been serially correlated with the risk of cardiovascular disease, and variants in the LPA gene have been found to be the strongest cardiovascular genetic risk factor among 2100 candidate genes for cardiovascular disease. Therefore, the etiologic role of Lp(a) in CVD has become a hot research topic.

Small interfering RNAs (siRNAs), based on the RNA interference mechanism, can inhibit or block the expression of target genes in a sequence-specific manner for the purpose of treating diseases. Therefore, by inhibiting the expression of the LPA gene, it is possible to prevent and treat diseases caused by abnormal lipoprotein(a) levels, especially cardiovascular diseases, at the cellular level. The Guidelines for the Prevention and Control of Dyslipidemia in Adults define >30 mg/dl as abnormal Lp(a), and using this criterion, approximately 30% of patients with previous cardiovascular events have abnormal Lp (a), and despite the fact that elevated Lp(a) levels are common, there is a lack of targeted therapeutic agents. Therefore, the development of an siRNA drug that inhibits LPA gene expression and is capable of alleviating, preventing and/or treating diseases associated with LPA gene expression is of great importance and could contribute to the reduction of adverse cardiovascular events.

### SUMMARY OF THE DISCLOSURE

The present disclosure provides a compound conjugated with an oligonucleotide for use in inhibiting expression of LPA gene, a pharmaceutical composition, and use thereof. The present disclosure provides a compound conjugated with an oligonucleotide and pharmaceutical compositions thereof capable of lowering cholesterol levels and thereby preventing or controlling metabolic syndrome. The compounds conjugated with oligonucleotides and the pharmaceutical compositions thereof provided by the present disclosure are useful for alleviating, preventing and/or treating a disease or condition mediated by dysregulation of LPA gene expression.

In a first aspect of the present disclosure, provided is a compound conjugated with an oligonucleotide having a structure represented by formula (I), or a pharmaceutically acceptable salt thereof:

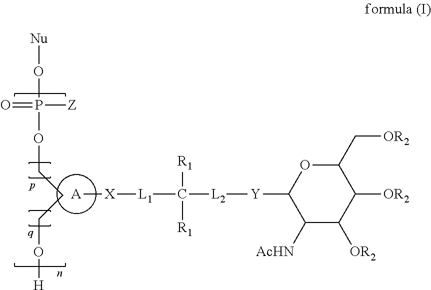

wherein, in the structure,
each A is independently an unsubstituted or substituted 4- to 10-membered aliphatic ring,
n is selected from the group consisting of 1, 2, 3 and 4,
each Z is independently selected from the group consisting of hydroxyl and mercapto,
each p is independently selected from the group consisting of 1, 2 and 3,
each q is independently selected from the group consisting of 1, 2 and 3,
each X is independently selected from the group consisting of NH, O and S,
each L₁ is independently selected from the group consisting of

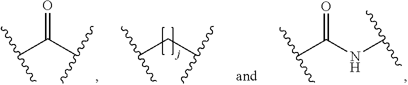

wherein j is selected from the group consisting of 1, 2, 3, 4, 5, 6, 7, 8, 9 and 10,
each R₁ is independently selected from the group consisting of H, C₁-C₆ alkyl, C₁-C₆ haloalkyl and C₁-C₆ alkoxy,
each L₂ is independently selected from the group consisting of C₁-C₃₀ alkylidene and

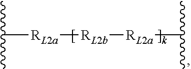

wherein each R_{L2a} is independently C₁-C₁₀ alkylidene, each R_{L2b} is independently selected from the group consisting of O, S, NH and —NH—C(O)—, and k is selected from the group consisting of 1, 2, 3, 4, 5, 6, 7, 8, 9 and 10,
each Y is independently selected from the group consisting of NH, O and S, and
each R₂ is independently selected from the group consisting of: H,

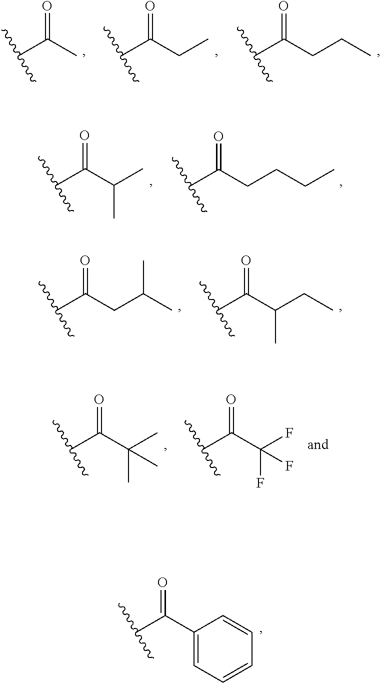

wherein Nu represents a double-stranded oligonucleotide or a pharmaceutically acceptable salt thereof for reducing the expression of intracellular LPA gene, the double-stranded oligonucleotide comprises a sense strand and an antisense strand, wherein the sense strand and the antisense strand form a double-stranded region, the antisense strand comprises a complementary region that has complementarity to a target sequence of LPA mRNA, and the target sequence is selected from a nucleotide region comprising 14 to 35 consecutive nucleotides on the LPA mRNA.

In some specific embodiments of the present disclosure, the sense strand of the double-stranded oligonucleotide represented by Nu comprises a nucleotide sequence of or differing by 1, 2 or 3 nucleotides from at least 17, at least 18, or at least 19 consecutive nucleotides in any one of the sequences set forth in SEQ ID NOs: 1, 3, 5, 7, 9, 11 and 13; and/or, the antisense strand comprises a nucleotide sequence of or differing by 1, 2 or 3 nucleotides from at least 17, at least 18, or at least 19 consecutive nucleotides in any one of the sequences set forth in SEQ ID NOs: 2, 4, 6, 8, 10, 12 and 14.

In some specific embodiments of the present disclosure, according to the 5′-3′direction, the antisense strand of the double-stranded oligonucleotide represented by Nu comprises a nucleotide sequence of or differing by 1, 2 or 3 nucleotides from 1 to 19 consecutive nucleotides in any one of the sequences set forth in SEQ ID NOs: 2, 4, 6, 8, 10, 12 and 14.

In some specific embodiments of the present disclosure, the antisense strand of the double-stranded oligonucleotide represented by Nu comprises a nucleotide sequence of or differing by 1 or 2 nucleotides from any one of the sequences set forth in SEQ ID NOs: 2, 4, 6, 8, 10, 12 and 14, and/or, the sense strand of the double-stranded oligonucleotide comprises a nucleotide sequence of or differing by 1 or 2 nucleotides from any one of the sequences set forth in SEQ ID NOs: 1, 3, 5, 7, 9, 11 and 13.

In some specific embodiments of the present disclosure, each nucleotide in the double-stranded oligonucleotide is independently selected from the group consisting of:

2′-fluoro modified nucleotide, 2′-deoxy modified nucleotide, 2′-O-methyl modified nucleotide, 2′-O—(CH₂)ₓ—O—Rₘ modified nucleotide, 2′-O—Si(Rₙ)₃ modified nucleotide, 2′-amino-modified nucleotide, abasic nucleotide, and a nucleotide analogue, wherein the nucleotide analogue is one or more selected from the group consisting of PNA, MNA, BNA, LNA, GNA, TNA and UNA.

Wherein, x is selected from the group consisting of 1 and 2, Rₘ is selected from the group consisting of optionally substituted C₁₋₆ alkyl and optionally substituted C₁₋₆ alkoxy, when Rₘ comprises a substituent, the substituent is selected from the group consisting of halogen, C₁₋₃ alkyl and C₁₋₃ alkoxy; Rₙ is independently selected from unsubstituted or optionally substituted C₁₋₆ alkyl, when Rₙ comprises a substituent, the substituent is selected from the group consisting of halogen, C₁₋₃ alkyl and C₁₋₃ alkoxy.

In some specific embodiments of the present disclosure, all nucleotides in the double-stranded oligonucleotide are independently selected from modified nucleotides, wherein the sense strand and the antisense strand are selected for modification:

In the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 9, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

In some specific embodiments of the present disclosure, all nucleotides in the double-stranded oligonucleotide are independently selected from modified nucleotides, wherein the sense strand and the antisense strand are selected for modification:

In the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 12, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

In some specific embodiments of the present disclosure, in the direction from the 5′ end to 3′ end, at least one of the following linkages is a phosphorothioate linkage: a linkage between the first nucleotide and the second nucleotide at 5′ end of the sense strand; and a linkage between the second nucleotide and the third nucleotide at 5′ end of the sense strand.

In some specific embodiments of the present disclosure, in the direction from the 5′ end to 3′ end, at least one of the following linkages is a phosphorothioate linkage: a linkage between the first nucleotide and the second nucleotide at 5′ end of the antisense strand; a linkage between the second nucleotide and the third nucleotide at 5′ end of the antisense strand; a linkage between the first nucleotide and the second nucleotide at 3′ end of the antisense strand; and a linkage between the second nucleotide and the third nucleotide at 3′ end of the antisense strand.

In some specific embodiments of the present disclosure, each nucleotide in the double-stranded oligonucleotide is a modified nucleotide.

The sense strand comprises or is selected from any one nucleotide sequence selected from the group consisting of modified nucleotide sequences set forth in A1) to A11).

The antisense strand comprises or is selected from any one nucleotide sequence selected from the group consisting of modified nucleotide sequences set forth in B1) to B41).

In a second aspect of the present disclosure, provided is a pharmaceutical composition, the pharmaceutical composition comprises the compound conjugated with an oligonucleotide according to the first aspect of the present disclosure.

In a third aspect of the present disclosure, provided is use of the compound conjugated with an oligonucleotide according to the first aspect of the present disclosure or the pharmaceutical composition according to the second aspect of the present disclosure in the manufacture of a medicament for alleviating, preventing and/or treating the LPA gene-mediated disease or condition.

In a fourth aspect of the present disclosure, provided is a kit, the kit comprises the compound conjugated with an oligonucleotide according to the first aspect of the present disclosure or the pharmaceutical composition according to the second aspect of the present disclosure.

In a fifth aspect of the present disclosure, provided is a method for inhibiting LPA gene expression in a subject in need thereof, wherein the method comprises administering to the subject the compound conjugated with an oligonucleotide according to the first aspect of the present disclosure or the pharmaceutical composition according to the second aspect of the present disclosure.

In a sixth aspect of the present disclosure, provided is a method for alleviating, treating and/or preventing a LPA-mediated disease or condition in a subject in need thereof, wherein the method comprises administering to the subject the compound conjugated with an oligonucleotide according to the first aspect of the present disclosure or the pharmaceutical composition according to the second aspect of the present disclosure.

In some specific embodiments of the present disclosure, the LPA gene-mediated disease or condition includes a disease related to mRNA expression level of LPA gene.

In some specific embodiments of the present disclosure, the LPA gene-mediated disease or condition includes cardiovascular disease, the cardiovascular disease includes but is not limited to hyperlipoproteinemia(a), Berger's disease, peripheral artery disease, coronary artery disease, metabolic syndrome, acute coronary syndrome, aortic stenosis, aortic regurgitation, aortic dissection, cerebrovascular disease, mesenteric ischemia, superior mesenteric artery occlusion, renal artery stenosis, stable/unstable angina pectoris, acute coronary syndrome, heterozygous or homozygous familial hypercholesterolemia, high apolipoprotein beta lipoprotein, cerebrovascular atherosclerosis, cerebrovascular disease and venous thrombosis, stroke, atherosclerosis, thrombosis, coronary artery disease and/or any other disease or condition related to the increased level of LP(a) particles.

The compound conjugated with an oligonucleotide and the pharmaceutical composition thereof show excellent LPA gene expression modulating activity in mouse model experiments. For example, the compound conjugated with an oligonucleotide and the pharmaceutical composition thereof provided by the present disclosure can significantly inhibit expression of LPA mRNA in animal liver tissue, and the effect level is better than that of Olpasiran. Therefore, the compound conjugated with an oligonucleotide and the pharmaceutical composition thereof provided by the present disclosure are useful for regulating the expression level of the target gene LPA, alleviating, preventing and/or treating a disease or condition mediated by the mRNA level of LPA gene expression, with good application prospects.

### BRIEF DESCRIPTION OF THE DRAWINGS

FIG. 1 shows the inhibition on target gene in BALB/c-HDI mice after administration of compounds conjugated with oligonucleotides in Example 1.

FIG. 2 shows the expression levels of Apo(a) protein in hApo(a) transgenic mice after administration of compounds conjugated with oligonucleotides in Example 2.

### DETAILED DESCRIPTION OF THE EXEMPLARY EMBODIMENTS

The present disclosure provides a compound conjugated with an oligonucleotide, a pharmaceutical composition and uses thereof. Those skilled in the art can learn from the content herein and appropriately improve the process parameters for realization. It should be particularly noted that all similar substitutions and modifications are obvious to those skilled in the art, and they are deemed to be included in the present disclosure. The method and the application of the present disclosure have been described through the preferred embodiments, and it is obvious that the method and application described herein may be changed or appropriately modified and combined to realize and apply the technology of the present disclosure by those skilled in the art without departing from the content, spirit and scope of the present disclosure.

### Term Explanation

As used herein, the terms “apolipoprotein(a) gene”, apo(a) gene and LPA are used interchangeably in the present disclosure. LPA includes but is not limited to human LPA, cynomolgus monkey LPA, mouse LPA and rat LPA. Its amino acid, complete coding sequence and mRNA sequence are easy to obtain by using the published database; Its amino acid, complete coding sequence and mRNA sequence can be natural, synthetic, recombinant or any combination of them.

As used herein, the term “including” or “comprising” is open-ended, which includes the contents specified in the present disclosure, and does not exclude other contents.

As used herein, “optional” or “optionally” means that the subsequently described event or condition may or may not occur, and that the description includes instances wherein the event or condition may or may not occur.

As used herein, the term “small interfering RNA (siRNA)” refers to a double-stranded RNA that comprises a sense and an antisense strands, and each strand is 17 to 30 nucleotides in length. The siRNA mediates the targeted cleavage of RNA transcripts via RNA-induced silencing complex (RISC) pathway through the formation of a RISC. Specifically, the siRNA directs the specific degradation of mRNA sequences through the known process of RNA interference (RNAi), which inhibits the translation of mRNA into amino acids and thereby transformation into proteins. For example, siRNA can regulate (e.g., inhibit) the expression of LPA in cells.

As used herein, the term “antisense strand (or called as guide strand)” includes a region that is substantially complementary to a target sequence (e.g., LPA mRNA). The term “sense strand (or called as passenger strand)” refers to an iRNA strand that comprises a region that is substantially complementary to the antisense strand. The term “substantially complementary” means fully complementary or at least partially complementary. For example, the antisense strand is fully complementary or at least partially complementary to a target sequence. In the case of partial complementarity, a mismatch may be present within the internal or end region of the molecule, wherein the most tolerated mismatch is present within the end region, e.g., within the 5, 4, 3 or 2 nucleotides at the 5′- and/or 3′-end of the iRNA.

It is noted that the antisense strand being “at least partially substantially complementary” to the mRNA means that the antisense strand has a polynucleotide that is substantially complementary to a contiguous portion of the mRNA of interest. Alternatively, when a polynucleotide is substantially non-intermittently complementary to a portion of the mRNA encoding LPA, the antisense strand is complementary to at least a portion of the LPA mRNA.

As used herein, the term “target sequence” refers to a contiguous portion of the nucleotide sequence of the mRNA molecule formed during transcription of the LPA gene, including mRNA that is the processed product of the primary transcription product RNA. LPA can be found in a cell, e.g., a cell in a subject.

As used herein, the term “complementary” refers to the ability of an oligonucleotide of a first sequence to hybridize with an oligonucleotide of a second sequence under certain conditions to form a double-stranded structure.

As used herein, the term “substantially complementary” means that there are not more than 3 nucleotide mispairings, not more than 2 nucleotide mispairings, or not more than 1 nucleotide mispairing between the sense and antisense strands in the double-stranded region, such as 3 nucleotide mispairings, 2 nucleotide mispairings, 1 nucleotide mispairing, and 0 nucleotide mispairing, while the ability to hybridize under the relevant conditions is retained. Additionally, in cases where one or more single-stranded protruding terminus formed when two oligonucleotides are designed for hybridization, such protruding terminus should not be considered as mispairings in terms of determining complementarity. In the present disclosure, the “complementary” sequence may also include, or be formed exclusively from non-Watson-Crick base pairs and/or from non-natural as well as modified nucleotides in terms of meeting the above hybridization capability requirements. Such non-Watson-Crick base pairs include, but are not limited to, G:U wobble base pair or Hoogstein base pair. Correspondingly, in the present disclosure, unless otherwise specified, “mispairing” means that in the siRNA duplex molecule, the bases at corresponding sites are not presented in a manner of being complementarily paired.

As used herein, the term “complementary sequence with complementarity” refers to the mismatch between the antisense chain of double-stranded oligonucleotides and the complementary sequence of LPA mRNA by no more than 3 nucleotides, no more than 2 nucleotides or no more than 1 nucleotide, such as 3 nucleotides, 2 nucleotides, 1 nucleotide or 0 nucleotide.

As used herein, the term “nucleotide difference”, the term “nucleotide base difference” and the term “nucleotide sequence difference” can be used interchangeably, which refers to a change in the base type of a nucleotide at the same or a corresponding position compared to the original nucleotide sequence. For example, if a nucleotide base in the original nucleotide sequence is A, and a nucleotide base at the same or corresponding position is changed to U, C, G, dT, dC, dG, or the like, it is considered that there is a difference in the nucleotide sequence at that position. It should be noted that in the case where the nucleotides at the same or corresponding positions differ only in the presence or absence of a modification or the type of modification as compared to the original nucleotide sequence, a difference in the nucleotide sequence at the position is not considered to exist.

As used herein, the term “protruding terminus” refers to at least one unpaired nucleotide protruding from the double helix structure of a double-stranded oligonucleotide, which is also a nucleotide sequence other than the double-stranded region in siRNA structure. For example, a nucleotide protruding terminus is present when the 3′ end of one strand of the sense and/or antisense strand extends beyond the 5′ end of the other strand, or when the 5′ end of one strand of the sense and/or antisense strand extends beyond the 3′ end of the other strand. The protruding terminus may comprise at least one nucleotide, at least two nucleotides, at least three nucleotides, at least four nucleotides, at least five nucleotides, or more nucleotides. The nucleotide protruding terminus may comprise, or consist of, a nucleotide/nucleoside analogue, including a deoxyribonucleotide/nucleoside. The protruding terminus may be located on the sense strand, the antisense strand, or any combination thereof. In addition, the nucleotide at the protruding terminus may present at the 5′ end, the 3′ end, or both ends of the antisense or the sense strand.

As used herein, the term “DEPC H2O” is ultrapure water (Type I water) treated with diethyl pyrocarbonate (DEPC) and sterilized under high temperature and high pressure.

As used herein, the term “subject” refers to any animal that is examined, studied, or treated, and it is not intended to limit the present disclosure to any particular type of subject. In some embodiments of the present disclosure, human is a preferred subject, while in some other embodiments, a non-human animal is a preferred subject, including, but not limited to, a mouse, monkey, ferret, cow, sheep, goat, pig, chicken, turkey, dog, cat, horse, and reptile.

As used herein, the term “inhibiting expression of LPA gene” includes any level of inhibition on LPA gene, e.g., at least partial inhibition on expression of LPA gene, such as inhibition by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. Wherein, the expression of LPA gene can be evaluated based on the level of any variable associated with the expression of LPA gene, e.g., mRNA levels or protein levels of LPA. Inhibition can be evaluated by a reduction in the absolute or relative level of one or more of these variables compared to control levels. The control level may be any type of control level utilized in the art, e.g., a baseline level before administration, or a measured level of a similar subject, cell, or sample that has not been treated or treated with a control (e.g., a control containing only buffer or control without containing an active ingredient).

As used herein, “conjugating” refers to two or more chemical moieties being linked to each other via a covalent linkage. A “conjugate” refers to a compound formed by covalent linkage of individual chemical moieties. A “conjugating molecule” can be understood as a specific compound capable of being conjugated with a siRNA via reactions, thus finally forming the oligonucleotide conjugate of the present disclosure.

As used herein, a “pharmaceutical composition” may be useful in the treatment of a disease or the in vitro cell culture. When used in the treatment of a disease, the term “pharmaceutical composition” generally is in a unit dose form and can be prepared by any of the methods known in the pharmaceutical field. All methods comprise a step of combining an active ingredient with one or more excipients as accessory ingredients. Typically, the composition is prepared by uniformly and sufficiently mixing the active siRNA with a liquid excipient, a finely divided solid excipient, or both.

As used herein, the term “pharmaceutically acceptable” means that the substance or composition must be chemically and/or toxicologically compatible with the other ingredients of the formulation and/or with the mammal being treated therewith. Preferably, “pharmaceutically acceptable” in the present disclosure means approved by a regulatory agency of the Federal or a state government or listed in the U.S. Pharmacopeia or other generally recognized pharmacopeia for use in animals, and particularly in humans.

As used herein, the term “pharmaceutically acceptable excipient” may include any solvent, solid excipient, diluent, other liquid excipient, etc., which is suitable for the specific target dosage form. Except to the extent that any conventional excipient is incompatible with the siRNA of the present disclosure, for example, producing any adverse biological effects or harmful interactions with any other components of a pharmaceutically acceptable composition, the uses thereof are also contemplated by the present disclosure.

As used herein, except to the extent that any conventional excipient is incompatible with the siRNA of the present disclosure, for example, producing any adverse biological effects or harmful interactions with any other components of a pharmaceutically acceptable composition, the uses thereof are also contemplated by the present disclosure.

Compound Conjugated with an Oligonucleotide

In a first aspect of the present disclosure, provided is a compound conjugated with an oligonucleotide or a pharmaceutically acceptable salt thereof, wherein the compound has a structure represented by formula (I):

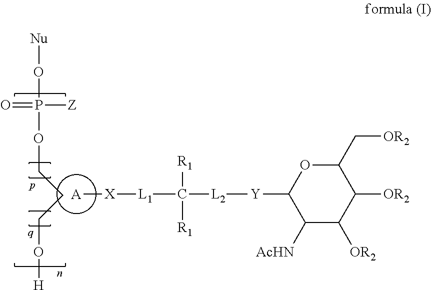

wherein, in the structure,
each A is independently an unsubstituted or substituted 4- to 10-membered aliphatic ring,
n is selected from the group consisting of 1, 2, 3 and 4,
each Z is independently selected from the group consisting of hydroxyl and mercapto (sulfydryl),
each p is independently selected from the group consisting of 1, 2 and 3,
each q is independently selected from the group consisting of 1, 2 and 3,
each X is independently selected from the group consisting of NH, O and S,
each L₁ is independently selected from the group consisting of

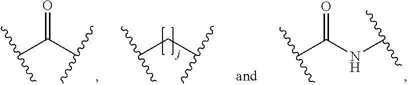

wherein j is selected from the group consisting of 1, 2, 3, 4, 5, 6, 7, 8, 9 and 10,
each R₁ is independently selected from the group consisting of H, C₁-C₆ alkyl, C₁-C₆ haloalkyl and C₁-C₆ alkoxy,
each L₂ is independently selected from the group consisting of C₁-C₃₀ alkylidene and

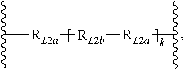

wherein each R_{L2a} is independently C₁-C₁₀ alkylidene, each R_{L2b} is independently selected from the group consisting of O, S, NH and —NH—C(O)—, and k is selected from the group consisting of 1, 2, 3, 4, 5, 6, 7, 8, 9 and 10,
each Y is independently selected from the group consisting of NH, O and S, and
each R₂ is independently selected from the group consisting of: H,

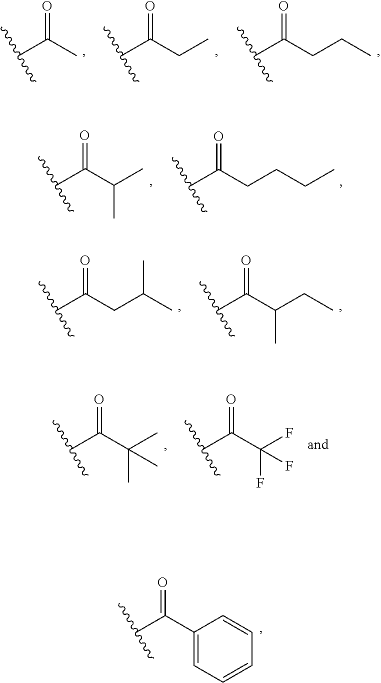

In some embodiments of the present disclosure, each A is independently selected from the group consisting of an unsubstituted or substituted 4- to 10-membered cycloalkane group and an unsubstituted or substituted 4- to 10-membered cycloolefin group.

In some embodiments of the present disclosure, each A is independently an unsubstituted or substituted 4- to 10-membered cycloalkane group.

In some embodiments of the present disclosure, each A is independently a 4- to 10-membered cycloalkane group, such as a monocyclic ring, a spirocyclic ring, or a bridged ring.

In some embodiments of the present disclosure, each A is independently selected from the group consisting of

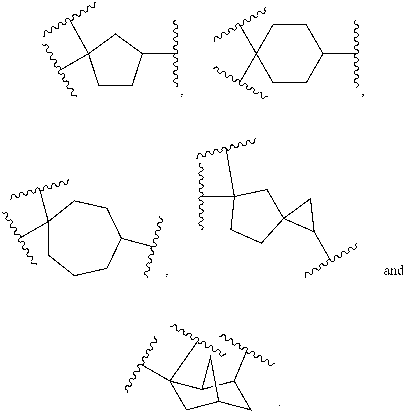 [SMILES: *C1CCC(*)(*)C1 ; *C1CCC(*)(*)CC1 ; *C1CCCC(*)(*)CC1 ; *C1CC12CCC(*)(*)C2 ; *C1CC2CC1C(*)(*)C2]

In some specific embodiments of the present disclosure, each A is

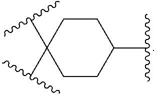 [SMILES: *C1CCC(*)(*)CC1]

In some specific embodiments of the present disclosure, the compound conjugated with an oligonucleotide has a structure represented by formula (II), or a pharmaceutically acceptable salt thereof:

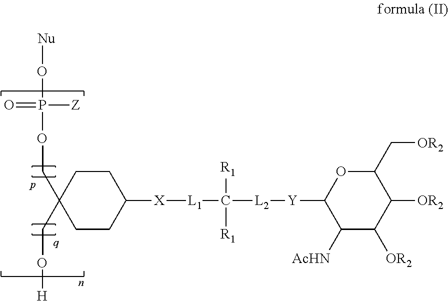

in formula (II), p, q, n, Z, X, Y, L₁, L₂ and R₁ are as defined above, and R₂ is H.

In some specific embodiments of the present disclosure, each X is NH.

In some specific embodiments of the present disclosure, each L₁ is

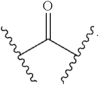 [SMILES: *C(*)=O]

In some specific embodiments of the present disclosure, the compound conjugated with an oligonucleotide has a structure represented by formula (III), or a pharmaceutically acceptable salt thereof:

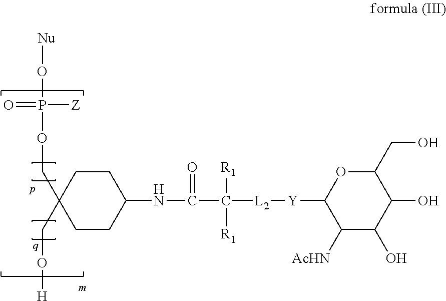

in formula (III), m is selected from the group consisting of 1, 2, 3 and 4, and the other substituents are as defined above.

In some specific embodiments of the present disclosure, each Z is hydroxyl.

In some embodiments of the present disclosure, each p is independently selected from the group consisting of 1 and 2.

In some specific embodiments of the present disclosure, each p is 1.

In some embodiments of the present disclosure, each q is independently selected from the group consisting of 1 and 2.

In some specific embodiments of the present disclosure, each q is 1.

In some specific embodiments of the present disclosure, each p is 1 and each q is 1.

In some specific embodiments of the present disclosure, each R₁ is H.

In some specific embodiments of the present disclosure, each Y is O.

Further, in some specific embodiments of the present disclosure, the compound conjugated with an oligonucleotide has a structure represented by formula (IV), or a pharmaceutically acceptable salt thereof,

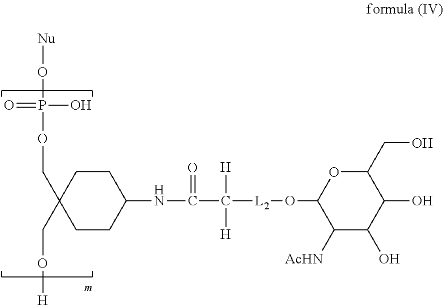

in formula (IV), m is selected from the group consisting of 1, 2, 3 and 4, and L₂ is independently selected from the group consisting of

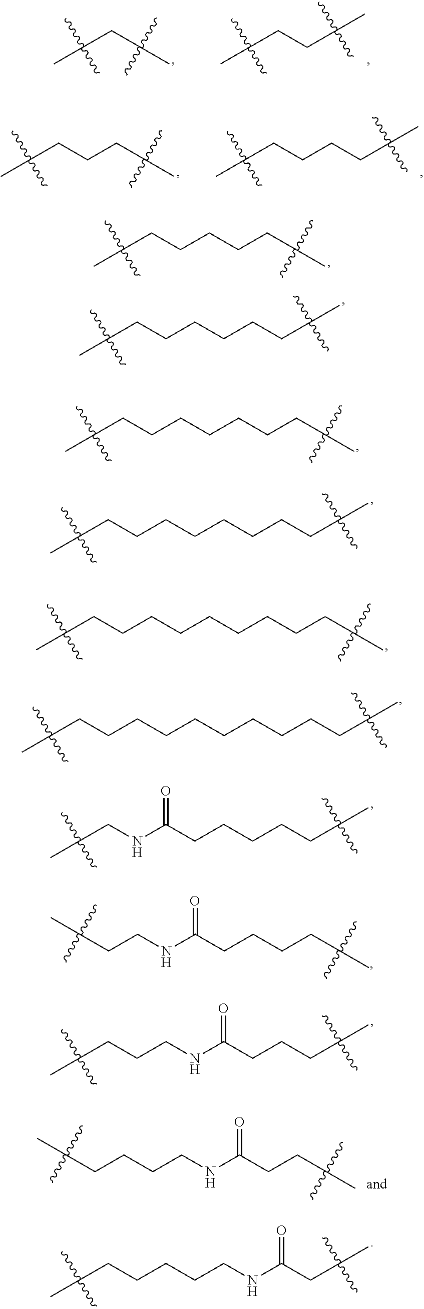

In some alternative embodiments of the present disclosure, each L₂ is independently selected from the group consisting of and

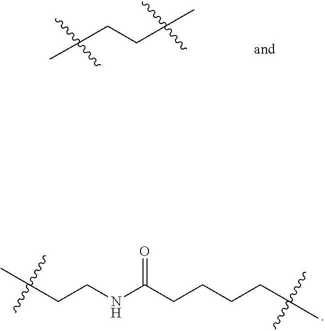

In some specific embodiments of the present disclosure, L₂ is

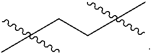 [SMILES: *CC*]

In some specific embodiments of the present disclosure, L₂ is

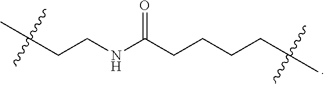 [SMILES: *CCCCC(=O)NCC*]

In some specific embodiments of the present disclosure, the compound conjugated with an oligonucleotide has a structure selected from the group consisting of:

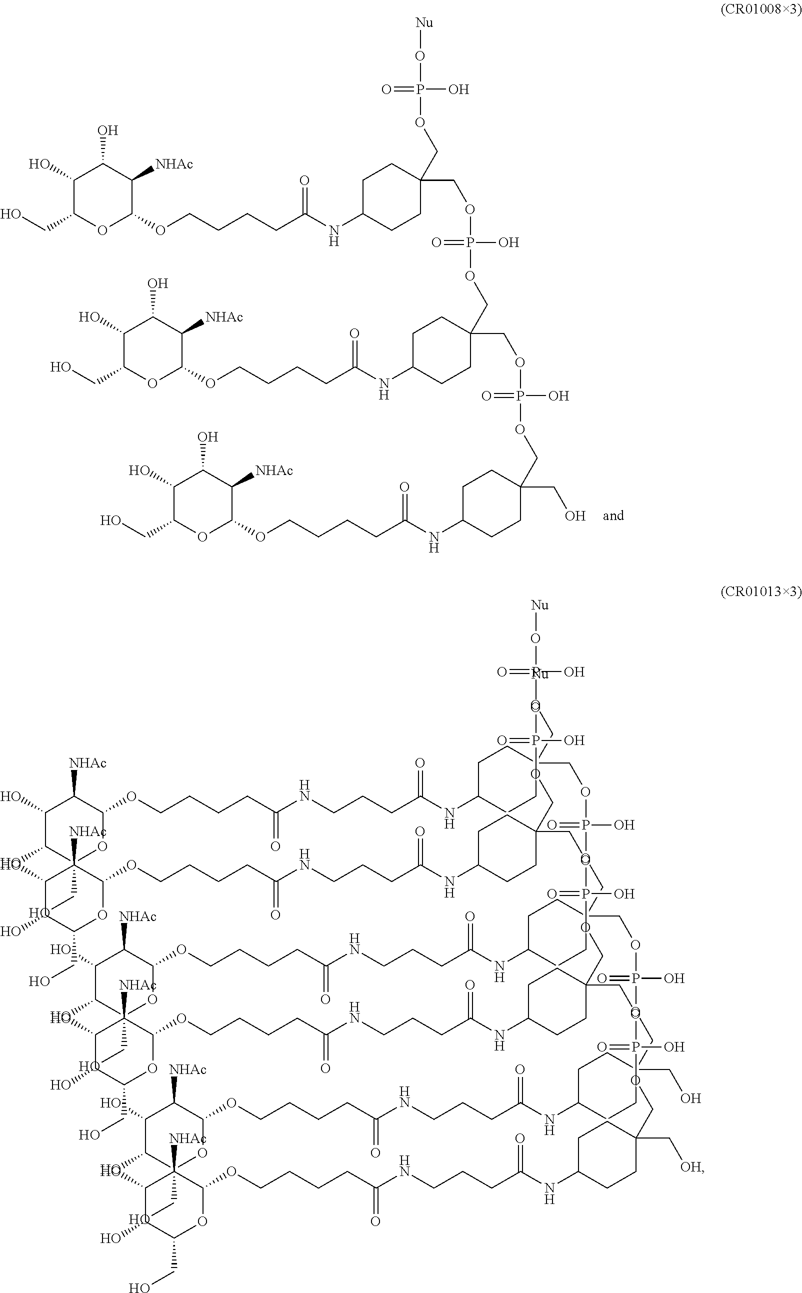

or a pharmaceutically acceptable salt thereof.

Wherein Nu represents a double-stranded oligonucleotide or a pharmaceutically acceptable salt thereof for reducing the expression of intracellular LPA gene, the double-stranded oligonucleotide comprises a sense strand and an antisense strand, wherein the sense strand and the antisense strand are complementary or substantially complementary to form a double-stranded region, and the “substantially complementary” means that there are not more than 3 nucleotide mispairings between the sense and antisense strands in the double-stranded region.

In the present disclosure, the antisense strand comprises a complementary region that has complementarity to a target sequence of LPA mRNA, and the target sequence is selected from a nucleotide region comprising 14 to 35 contiguous nucleotides on the LPA mRNA.

Further, in the direction from 5′ end to 3′ end, nucleotides at positions 2 to 19 of the antisense strand comprise a complementary region that has complementarity to a target sequence of LPA mRNA.

In some specific embodiments of the present disclosure, the sense strand of Nu in the compound conjugated with an oligonucleotide is linked to a phosphate group at 3′ end.

In some specific embodiments of the present disclosure, the sense strand of the double-stranded oligonucleotide represented by Nu comprises a nucleotide sequence of or differing by 1, 2 or 3 nucleotides from at least 17, at least 18, or at least 19 consecutive nucleotides in any one of the sequences set forth in SEQ ID NOs.1, 3, 5, 7, 9, 11 and 13; and/or, the antisense strand comprises a nucleotide sequence of or differing by 1, 2 or 3 nucleotides from at least 17, at least 18, or at least 19 consecutive nucleotides in any one of the sequences set forth in SEQ ID NOs.2, 4, 6, 8, 10, 12 and 14.

In some specific embodiments of the present disclosure, according to the 5′-3′direction, the antisense strand of the double-stranded oligonucleotide represented by Nu comprises a nucleotide sequence of or differing by 1, 2 or 3 nucleotides from 1st to 19th consecutive nucleotides in any one of the sequences set forth in SEQ ID NOs.2, 4, 6, 8, 10, 12 and 14.

In some specific embodiments of the present disclosure, the antisense strand of the double-stranded oligonucleotide represented by Nu comprises a nucleotide sequence of or differing by 1 or 2 nucleotides from any one of the sequences set forth in SEQ ID NOs.2, 4, 6, 8, 10, 12 and 14.

In some specific embodiments of the present disclosure, the sense strand of the double-stranded oligonucleotide represented by Nu comprises a nucleotide sequence of or differing by 1 or 2 nucleotides from any one of the sequences set forth in SEQ ID NOs.1, 3, 5, 7, 9, 11 and 13.

In some specific embodiments of the present disclosure, the antisense strand of the double-stranded oligonucleotide represented by Nu comprises a nucleotide sequence selected from the group consisting of the sequences set forth in SEQ ID NOs.2, 4, 6, 8, 10, 12 and 14, and the sense strand of the double-stranded oligonucleotide represented by Nu comprises a nucleotide sequence selected from the group consisting of the sequences set forth in SEQ ID NOs.1, 3, 5, 7, 9, 11 and 13.

In some specific embodiments of the present disclosure, the double-stranded oligonucleotide is one or more selected from the group consisting of:
1) an antisense strand having a nucleotide sequence of or differing by 1 or 2 nucleotides from the nucleotide sequence set forth in SEQ ID NO.2 and a sense strand having a nucleotide sequence of or differing by 1 or 2 nucleotides from the nucleotide sequence set forth in SEQ ID NO.1;
2) an antisense strand having a nucleotide sequence of or differing by 1 or 2 nucleotides from the nucleotide sequence set forth in SEQ ID NO.4 and a sense strand having a nucleotide sequence of or differing by 1 or 2 nucleotides from the nucleotide sequence set forth in SEQ ID NO.3;
3) an antisense strand having a nucleotide sequence of or differing by 1 or 2 nucleotides from the nucleotide sequence set forth in SEQ ID NO.6 and a sense strand having a nucleotide sequence of or differing by 1 or 2 nucleotides from the nucleotide sequence set forth in SEQ ID NO.5;
4) an antisense strand having a nucleotide sequence of or differing by 1 or 2 nucleotides from the nucleotide sequence set forth in SEQ ID NO.8 and a sense strand having a nucleotide sequence of or differing by 1 or 2 nucleotides from the nucleotide sequence set forth in SEQ ID NO.7;
5) an antisense strand having a nucleotide sequence of or differing by 1 or 2 nucleotides from the nucleotide sequence set forth in SEQ ID NO.10 and a sense strand having a nucleotide sequence of or differing by 1 or 2 nucleotides from the nucleotide sequence set forth in SEQ ID NO.9;
6) an antisense strand having a nucleotide sequence of or differing by 1 or 2 nucleotides from the nucleotide sequence set forth in SEQ ID NO.12 and a sense strand having a nucleotide sequence of or differing by 1 or 2 nucleotides from the nucleotide sequence set forth in SEQ ID NO.11; and
7) an antisense strand having a nucleotide sequence of or differing by 1 or 2 nucleotides from the nucleotide sequence set forth in SEQ ID NO.14 and a sense strand having a nucleotide sequence of or differing by 1 or 2 nucleotides from the nucleotide sequence set forth in SEQ ID NO.13.

In some specific embodiments of the present disclosure, the double-stranded oligonucleotide is one or more selected from the group consisting of:
1) an antisense strand having a nucleotide sequence set forth in SEQ ID NO.2 and a sense strand having a nucleotide sequence set forth in SEQ ID NO.1;
2) an antisense strand having a nucleotide sequence set forth in SEQ ID NO.4 and a sense strand having a nucleotide sequence set forth in SEQ ID NO.3;
3) an antisense strand having a nucleotide sequence set forth in SEQ ID NO.6 and a sense strand having a nucleotide sequence set forth in SEQ ID NO.5;
4) an antisense strand having a nucleotide sequence set forth in SEQ ID NO.8 and a sense strand having a nucleotide sequence set forth in SEQ ID NO.7;
5) an antisense strand having a nucleotide sequence set forth in SEQ ID NO.10 and a sense strand having a nucleotide sequence set forth in SEQ ID NO.9;
6) an antisense strand having a nucleotide sequence set forth in SEQ ID NO.12 and a sense strand having a nucleotide sequence set forth in SEQ ID NO.11; and
7) an antisense strand having a nucleotide sequence set forth in SEQ ID NO.14 and a sense strand having a nucleotide sequence set forth in SEQ ID NO.13.

In some embodiments of the present disclosure, the siRNA may also contain modified nucleotides as required, and the modified nucleotides have no effect of weakening or invalidating the function of the siRNA to inhibit expression of LPA gene. At present, there are many ways to modify siRNA in this field, including, for example, backbone modification (such as phosphate group modification), ribose group modification and base modification. In some embodiments of the present disclosure, at least one nucleotide in the sense strand or antisense strand of the siRNA is a modified nucleotide, for example, the modified nucleotide is a nucleotide group in which a ribose group and an optional phosphate group are modified, but not limited thereto. In some specific embodiments of the present disclosure, each nucleotide in the double-stranded oligonucleotide is independently a modified or unmodified nucleotide.

In some specific embodiments of the present disclosure, substantially all nucleotides in the double-stranded oligonucleotide are independently selected from modified nucleotides. Wherein, “substantially all nucleotides are selected from modified nucleotides” means that most but not all of the nucleotides in the double-stranded oligonucleotide are modified, and the double-stranded oligonucleotide may contain no more than 5, 4, 3, 2 or 1 unmodified nucleotide.

In some specific embodiments of the present disclosure, all nucleotides in the double-stranded oligonucleotide are independently selected from modified nucleotides.

Wherein the nucleotide has a structure of

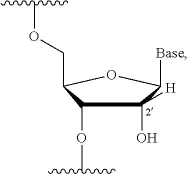

Base represents a nucleoside base, and the nucleoside base on each nucleotide is independently selected from the group consisting of uracil U, thymine T, cytosine C, adenine A, and guanine G.

In some specific embodiments of the present disclosure, each nucleotide in the double-stranded oligonucleotide is independently one or more selected from the group consisting of:

2′-fluoro modified nucleotide, 2′-deoxy modified nucleotide, 2′-O-methyl modified nucleotide, 2′-O—(CH₂)ₓ—O—Rₘ modified nucleotide, 2′-O—Si(Rₙ)₃ modified nucleotide, 2′-amino-modified nucleotide, abasic nucleotide, and a nucleotide analogue, wherein the nucleotide analogue is one or more selected from the group consisting of peptide nucleic acid (PNA), morpholino (MNA), bridged nucleic acid (BNA), locked nucleic acid (LNA), glycol nucleic acid (GNA), threose nucleic acid (TNA) and unlocked nucleic acid (UNA).

Wherein, x is selected from the group consisting of 1 and 2, Rₘ is selected from the group consisting of optionally substituted C₁₋₆ alkyl and optionally substituted C₁₋₆ alkoxy, when Rₘ comprises a substituent, the substituent is selected from the group consisting of halogen, C₁₋₆ alkoxy, hydroxyl and amino.

Rₐ is independently selected from unsubstituted or optionally substituted C₁₋₆ alkyl, when Rₐ comprises a substituent, the substituent is selected from the group consisting of halogen, C₁₋₃ alkyl and C₁₋₃ alkoxy.

In the present disclosure, a 2′-O—(CH₂)ₓ—O—Rₘ modified nucleotide means that a hydrogen atom on the hydroxyl group at the 2′position of the ribosyl group of the nucleotide is substituted by —(CH₂)ₓ—Rₘ. Wherein, when x is 1, the 2′-O—(CH₂)ₓ—O—Rₘ modified nucleotide is selected from the group consisting of a 2′-O-ethoxymethyl-modified nucleotide and a 2′-O-2,2,2-trifluoroethoxymethyl-modified nucleotide. When x is 2, the 2′-O—(CH₂)ₓ—O—Rₘ modified nucleotide is a 2′-O-methoxyethyl modified nucleotide (also known as a 2′-O-moe modified nucleotide).

In some specific embodiments of the present disclosure, the 2′-O—(CH₂)ₓ—O—Rₘ modified nucleotide is selected from the group consisting of a 2′-O-methoxyethyl-modified nucleotide and a 2′-O-ethoxymethyl-modified nucleotide.

In the present disclosure, a 2′-O—Si(Rₙ)₃ modified nucleotide means that a hydrogen atom on the hydroxyl group at the 2′position of the ribosyl group of the nucleotide is substituted by —Si(Rₙ)₃. For example, 2′-O-TBDMS modified nucleotide, 2′-O-TIPS modified nucleotide or 2′-O-TOM modified nucleotide;

wherein the structural formula of TBDMS is

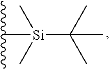 [SMILES: *[Si](C)(C)C(C)(C)C]

the structural formula of TIPS is

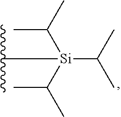 [SMILES: *[Si](C(C)C)(C(C)C)C(C)C]

and the structural formula of TOM is

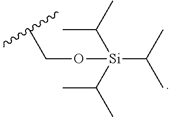 [SMILES: *CO[Si](C(C)C)(C(C)C)C(C)C]

In some specific embodiments of the present disclosure, the 2′-O—Si(Rₙ)₃ modified nucleotide is selected from the group consisting of a 2′-O-TBDMS modified nucleotide, a 2′-O-TIPS modified nucleotide and a 2′-O-TOM modified nucleotide.

In some specific embodiments of the present disclosure, the double-stranded oligonucleotide comprises at least one 2′-O-methoxyethyl modified nucleotide.

In some specific embodiments of the present disclosure, the sense strand is 18, 19, 20 or 21nt in length and the antisense strand is 19, 20, 21, 22 or 23 nt in length.

In some specific embodiments of the present disclosure, the sense strand is 19 nt in length and the antisense strand is 21 nt in length.

In some specific embodiments of the present disclosure, the antisense strand comprises at least one 2′-O-methoxyethyl modified nucleotide.

In some specific embodiments of the present disclosure, the antisense strand comprises at most two 2′-O-methoxyethyl modified nucleotides.

In some specific embodiments of the present disclosure, in the direction from 5′ end to 3′ end, at least three nucleotides at positions 7 to 10 of the nucleotide sequence of the sense strand of the double-stranded oligonucleotide are 2′-fluoro modified nucleotides, and nucleotides at the other positions in the sense strand are other than 2′-fluoro modified nucleotides; nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, and nucleotides at the other positions in the antisense strand are other than 2′-fluoro modified nucleotides.

In some specific embodiments of the present disclosure, in the direction from 5′ end to 3′ end, at least three nucleotides at positions 7 to 10 of the nucleotide sequence of the sense strand of the double-stranded oligonucleotide are 2′-fluoro modified nucleotides, and nucleotides at the other positions in the sense strand are 2′-O-methyl modified nucleotides or 2′-O-methoxyethyl modified nucleotides; nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, and nucleotides at the other positions in the antisense strand are 2′-O-methyl modified nucleotides or 2′-O-methoxyethyl modified nucleotides.

In some specific embodiments of the present disclosure, in the direction from 5′ end to 3′ end, at least three nucleotides at positions 7 to 10 of the nucleotide sequence of the sense strand of the double-stranded oligonucleotide are 2′-fluoro modified nucleotides, at most two nucleotides at positions 5, 12, 18 are 2′-O-methoxyethyl modified nucleotides, and nucleotides at the other positions in the sense strand are 2′-O-methyl modified nucleotides; nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, a nucleotide at position 15 is a 2′-O-methoxyethyl-modified nucleotide, and nucleotides at the other positions in the antisense strand are 2′-O-methyl modified nucleotides.

In some specific embodiments of the present disclosure, in the direction from 5′ end to 3′ end, at least three nucleotides at positions 7 to 10 of the nucleotide sequence of the sense strand of the double-stranded oligonucleotide are 2′-fluoro modified nucleotides, at most two nucleotides at positions 5, 12, 18 are 2′-O-methoxyethyl modified nucleotides, and nucleotides at the other positions in the sense strand are 2′-O-methyl modified nucleotides; nucleotides at positions 2, 6, 11, 14, and 16 of the nucleotide sequence of the antisense strand are 2′-fluoro modified nucleotides, a nucleotide at position 15 is a 2′-O-methoxyethyl-modified nucleotide, and nucleotides at the other positions in the antisense strand are 2′-O-methyl modified nucleotides.

In some specific embodiments of the present disclosure, in the direction from 5′ end to 3′ end, at least three nucleotides at positions 7 to 10 of the nucleotide sequence of the sense strand of the double-stranded oligonucleotide are 2′-fluoro modified nucleotides, and nucleotides at the other positions in the sense strand are 2′-O-methyl modified nucleotides; nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, a nucleotide at position 15 is a 2′-O-methoxyethyl-modified nucleotide, and nucleotides at the other positions in the antisense strand are 2′-O-methyl modified nucleotides.

In some specific embodiments of the present disclosure, in the direction from 5′ end to 3′ end, nucleotides at positions 7 to 10 of the nucleotide sequence of the sense strand of the double-stranded oligonucleotide are 2′-fluoro modified nucleotides, and nucleotides at the other positions in the sense strand are 2′-O-methyl modified nucleotides; nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, a nucleotide at position 15 is a 2′-O-methoxyethyl-modified nucleotide, and nucleotides at the other positions in the antisense strand are 2′-O-methyl modified nucleotides.

In some specific embodiments of the present disclosure, the sense strand and the antisense strand in the double-stranded oligonucleotide have a modification selected from the group consisting of (1) to (10):

(1) in the direction from 5′ end to 3′ end, in the sense strand, nucleotides at positions 7 to 9 of the nucleotide sequence are 2′-fluoro modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, and nucleotides at the other positions are 2′-O-methyl-modified nucleotides.

(2) in the direction from 5′ end to 3′ end, in the sense strand, nucleotides at positions 7 to 9 of the nucleotide sequence are 2′-fluoro modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(3) in the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, and nucleotides at the other positions are 2′-O-methyl-modified nucleotides.

(4) in the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 9, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(5) in the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 10, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(6) in the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(7) in the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 12, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(8) in the direction from 5′ end to 3′ end, in the sense strand, a nucleotide at position 5 is a 2′-O-methoxyethyl-modified nucleotide, nucleotides at positions 7 to 9 of the nucleotide sequence are 2′-fluoro modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(9) in the direction from 5′ end to 3′ end, in the sense strand, a nucleotide at position 5 is a 2′-O-methoxyethyl-modified nucleotide, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

(10) in the direction from 5′ end to 3′ end, in the sense strand, nucleotides at positions 7 to 10 of the nucleotide sequence are 2′-fluoro modified nucleotides, nucleotides at positions 12 and 18 are 2′-O-methoxyethyl-modified nucleotides, and nucleotides at the other positions are 2′-O-methyl modified nucleotides; in the antisense strand, nucleotides at positions 2, 6, 11, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

In some specific embodiments of the present disclosure, all nucleotides in the double-stranded oligonucleotide are independently selected from modified nucleotides, wherein the sense strand and the antisense strand are selected for modification:

In the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 9, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

In some specific embodiments of the present disclosure, all nucleotides in the double-stranded oligonucleotide are independently selected from modified nucleotides, wherein the sense strand and the antisense strand are selected for modification:

In the direction from 5′ end to 3′ end, in the sense strand, nucleotides at positions 7 to 10 of the nucleotide sequence are 2′-fluoro modified nucleotides, and nucleotides at the other positions are 2′-O-methyl modified nucleotides; in the antisense strand, nucleotides at positions 2, 6, 12, 14, and 16 of the nucleotide sequence are 2′-fluoro modified nucleotides, a nucleotide at position 15 is a 2′-O-methoxyethyl-modified nucleotide, and nucleotides at the other positions are 2′-O-methyl-modified nucleotides.

In some specific embodiments of the present disclosure, the sense strand and/or the antisense strand independently comprise one or more phosphorothioate linkages between nucleotides.

In some specific embodiments of the present disclosure, in the direction from the 5′ end to 3′ end, at least one of the following linkages is a phosphorothioate linkage:
(1) a linkage between the first nucleotide and the second nucleotide at 5′ end of the sense strand, and
(2) a linkage between the second nucleotide and the third nucleotide at 5′ end of the sense strand.

In some specific embodiments of the present disclosure, in the direction from the 5′ end to 3′ end, at least one of the following linkages is a phosphorothioate linkage:
(1) a linkage between the first nucleotide and the second nucleotide at 5′ end of the antisense strand,
(2) a linkage between the second nucleotide and the third nucleotide at 5′ end of the antisense strand,
(3) a linkage between the first nucleotide and the second nucleotide at 3′ end of the antisense strand, and
(4) a linkage between the second nucleotide and the third nucleotide at 3′ end of the antisense strand.

In some optional embodiments of the present disclosure, the sense strand or the antisense strand comprises a 3′ protruding terminus having at least 1 nucleotide.

In some optional embodiments of the present disclosure, the antisense strand comprises a 3′ protruding terminus having at least 1 nucleotide.

In some optional embodiments of the present disclosure, the antisense strand comprises a 3′ protruding terminus having 2 nucleotides.

In some specific embodiments of the present disclosure, each nucleotide in the double-stranded oligonucleotide is a modified nucleotide.

In some specific embodiments of the present disclosure, the sense strand comprises or is selected from any one nucleotide sequence selected from the group consisting of modified nucleotide sequences set forth in A1) to A11):
in the direction from the 5′ end to 3′ end of the sense strand:

A1:
GmsGmsCmUmUmGmAfUfCfAfUmGmAmAmCmUmAmCmUm;
A2:
GmsCmsAmGmCmUmCfCfUfUfAmUmUmGmUmUmAmUmAm;
A3:
GmsCmsUmCmCmUmUfAfUfUfGmUmUmAmUmAmCmGmAm;
A4:
CmsGmsGmUmAmAmUfGfGfAfCmAmGmAmGmUmUmAmUm;
A5:
CmsUmsGmGmCmUmUfGfAfUfCmAmAmGmAmAmCmUmAm;
A6:
AmsGmsAmGmGmAmCfAfAfCmAmGmAmAmUmAmUmUmAm;
A7:
AmsGmsAmGmG(moe)AmCfAfAfCmAmGmAmAmUmAmUmUmAm;
A8:
AmsGmsAmGmGmAmCfAfAfCfAmGmAmAmUmAmUmUmAm;
A9:
CmsUmsGmAmCmAmCfAfAfUfGmCmUmCmAmGmAmAmAm;
A10:
CmsUmsGmAmC(moe)AmCfAfAfUfGmCmUmCmAmGmAmAmAm;
A11:
CmsUmsGmAmCmAmCfAfAfUfGmC(moe)UmCmAmGmAmA(moe)
Am;

In some specific embodiments of the present disclosure, the antisense strand comprises or is selected from any one nucleotide sequence selected from the group consisting of modified nucleotide sequences set forth in B1) to B41):
in the direction from the 5′ end to 3′ end of the antisense strand:

B1:
AmsGfsUmAmGmUfUmCmAmUmGfAmUmCfAmAfGmCmCmsAmsGm;
B2:
AmsGfsUmAmGmUfUmCmAmUmGfAmUmCfA(moe)AfGmCmCmsAmsGm;
B3:
UmsAfsUmAmAmCfAmAmUmAmAfGmGmAfGmCfUmGmCmsCmsAm;
B4:
UmsAfsUmAmAmCfAmAmUmAmAfGmGmAfG(moe)CfUmGmCmsCmsAm;
B5:
UmsCfsGmUmAmUfAmAmCmAmAfUmAmAfGmGfAmGmCmsUmsGm;
B6:
AmsUfsAmAmCmUfCmUmGmUmCfCmAmUfUmAfCmCmGmsUmsGm;
B7:
AmsUfsAmAmCmUfCmUmGmUmCfCmAmUfT(moe)AfCmCmGmsUmsGm;
B8:
UmsAfsGmUmUmCfUmUmGmAmUfCmAmAfGmCfCmAmGmsCmsAm;
B9:
UmsAfsGmUmUmCfUmUmGmAmUfCmAmAfG(moe)CfCmAmGmsCmsAm;
B10:
UmsAfsAmUmAmUfUmCmUmGmUfUmGmUfCmCfUmCmUmsGmsAm;
B11:
UmsAfsAmUmAmUfUmCmUmGmUfUmGmUfC(moe)CfUmCmUmsGmsAm;
B12:
UmsUfsUmCmUmGfAmGmCmAmUfUmGmUfGmUfCmAmGmsAmsUm;
B13:
UmsUfsUmCmUmGfAmGmCmAmUfUmGmUfG(moe)UfCmAmGmsAmsUm;
B14:
AmsGfsUmAmGmUfUmCmAfUmGmAmUmCfAmAfGmCmCmsAmsGm;
B15:
AmsGfsUmAmGmUfUmCmAmUmGmAfUmCfAmAfGmCmCmsAmsGm;
B16:
AmsGfsUmAmGmUfUmCmAfUmGmAmUmCfA(moe)AfGmCmCmsAmsGm;
B17:
AmsGfsUmAmGmUfUmCmAmUmGmAfUmCfA(moe)AfGmCmCmsAmsGm;
B18:
UmsAfsUmAmAmCfAmAmUfAmAmGmGmAfGmCfUmGmCmsCmsAm;
B19:
UmsAfsUmAmAmCfAmAmUmAmAmGfGmAfGmCfUmGmCmsCmsAm;
B20:
UmsAfsUmAmAmCfAmAmUfAmAmGmGmAfG(moe)CfUmGmCmsCmsAm;
B21:
UmsAfsUmAmAmCfAmAmUmAmAmGfGmAfG(moe)CfUmGmCmsCmsAm;
B22:
UmsCfsGmUmAmUfAmAmCfAmAmUmAmAfGmGfAmGmCmsUmsGm;
B23:
UmsCfsGmUmAmUfAmAmCmAmAmUfAmAfGmGfAmGmCmsUmsGm;
B24:
UmsCfsGmUmAmUfAmAmCfAmAmUmAmAfG(moe)GfAmGmCmsUmsGm;
B25:
UmsCfsGmUmAmUfAmAmCmAmAmUfAmAfG(moe)GfAmGmCmsUmsGm;
B26:
AmsUfsAmAmCmUfCmUmGfUmCmCmAmUfUmAfCmCmGmsUmsGm;
B27:
AmsUfsAmAmCmUfCmUmGmUmCmCfAmUfUmAfCmCmGmsUmsGm;
B28:
AmsUfsAmAmCmUfCmUmGfUmCmCmAmUfU(moe)AfCmCmGmsUmsGm;
B29:
AmsUfsAmAmCmUfCmUmGmUmCmCfAmUfU(moe)AfCmCmGmsUmsGm;
B30:
UmsAfsGmUmUmCfUmUmGfAmUmCmAmAfGmCfCmAmGmsCmsAm;
B31:
UmsAfsGmUmUmCfUmUmGmAmUmCfAmAfGmCfCmAmGmsCmsAm;
B32:
UmsAfsGmUmUmCfUmUmGfAmUmCmAmAfG(moe)CfCmAmGmsCmsAm;
B33:
UmsAfsGmUmUmCfUmUmGmAmUmCfAmAfG(moe)CfCmAmGmsCmsAm;
B34:
UmsAfsAmUmAmUfUmCmUfGmUmUmGmUfCmCfUmCmUmsGmsAm;
B35:
UmsAfsAmUmAmUfUmCmUmGmUmUfGmUfCmCfUmCmUmsGmsAm;
B36:
UmsAfsAmUmAmUfUmCmUfGmUmUmGmUfC(moe)CfUmCmUmsGmsAm;
B37:
UmsAfsAmUmAmUfUmCmUmGmUmUfGmUfC(moe)CfUmCmUmsGmsAm;
B38:
UmsUfsUmCmUmGfAmGmCfAmUmUmGmUfGmUfCmAmGmsAmsUm;
B39:
UmsUfsUmCmUmGfAmGmCmAmUmUfGmUfGmUfCmAmGmsAmsUm;
B40:
UmsUfsUmCmUmGfAmGmCfAmUmUmGmUfG(moe)UfCmAmGmsAmsUm;
B41:
UmsUfsUmCmUmGfAmGmCmAmUmUfGmUfG(moe)UfCmAmGmsAmsUm;

Wherein C represents cytidine-3′-phosphate, G represents guanosine-3′-phosphate, U represents uridine-3′-phosphate, A represents adenosine-3′-phosphate, and T represents thymidine-3′-phosphate, m represents that the nucleotide adjacent to the left side of the letter m is a 2′-O-methyl-modified nucleotide, f represents that the nucleotide adjacent to the left side of the letter f is a 2′-fluoro-modified nucleotide, (moe) represents that the nucleotide adjacent to the left side of the combination sign (moe) is a 2′-O-methoxyethyl-modified nucleotide, and s represents that the two nucleotides adjacent to both sides of the letter s are linked by a phosphorothioate linkage.

In some specific embodiments of the present disclosure, the double-stranded oligonucleotide is one or more sets selected from the group consisting of: set 1, set 2, set 3, set 4, set 5, set 6, set 7 and set 8.

| | sense strand (5′-3′) | antisense strand (5′-3′) |
|---|---|---|
| set 1 | GmsGmsCmUmUmGmAfUfCf | AmsGfsUmAmGmUfUmCmAmUmGfAm |
| | AfUmGmAmAmCmUmAmCmUm | UmCfAmAfGmCmCmsAmsGm |
| set 2 | GmsGmsCmUmUmGmAfUfCf | AmsGfsUmAmGmUfUmCmAmUmGfAm |
| | AfUmGmAmAmCmUmAmCmUm | UmCfA(moe)AfGmCmCmsAmsGm |
| set 3 | GmsCmsAmGmCmUmCfCfUf | UmsAfsUmAmAmCfAmAmUmAmAfGm |
| | UfAmUmUmGmUmUmAmUmAm | GmAfGmCfUmGmCmsCmsAm |
| set 4 | GmsCmsAmGmCmUmCfCfUf | UmsAfsUmAmAmCfAmAmUmAmAfGm |
| | UfAmUmUmGmUmUmAmUmAm | GmAfG(moe)CfUmGmCmsCmsAm |
| set 5 | GmsCmsUmCmCmUmUfAfUf | UmsCfsGmUmAmUfAmAmCmAmAfUm |
| | UfGmUmUmAmUmAmCmGmAm | AmAfGmGfAmGmCmsUmsGm |
| set 6 | CmsUmsGmGmCmUmUfGfAf | UmsAfsGmUmUmCfUmUmGmAmUfCm |
| | UfCmAmAmGmAmAmCmUmAm | AmAfGmCfCmAmGmsCmsAm |
| set 7 | CmsUmsGmGmCmUmUfGfAf | UmsAfsGmUmUmCfUmUmGmAmUfCm |
| | UfCmAmAmGmAmAmCmUmAm | AmAfG(moe)CfCmAmGmsCmsAm |
| set 8 | CmsUmsGmAmCmAmCfAfAf | UmsUfsUmCmUmGfAmGmCmAmUfUm |
| | UfGmCmUmCmAmGmAmAmAm | GmUfG(moe)UfCmAmGmsAmsUm |

In some specific embodiments of the present disclosure, the double-stranded oligonucleotide comprises a set 2.

| set | sense strand | antisense strand |
|---|---|---|
| | (5′-3′) | (5′-3′) |
| set 2 | GmsGmsCmUmUmGm | AmsGfsUmAmGmUfUmCmAmU |
| | AfUfCfAfUmGmAm | mGfAmUmCfA(moe)AfGmCm |
| | AmCmUmAmCmUm | CmsAmsGm |

In some specific embodiments ofthe present disclosure, the compound conjugated with an oligonucleotide is selected from one or more of the groups shown in Table 6.

In some specific embodiments of the present disclosure, the compound conjugated with an oligonucleotide is selected from one or more of the following groups:

| | sense strand _(5′-3′) | antisense strand _(5′-3′) |
|---|---|---|
| RZ001031 | GmsGmsCmUmUmGmAfUfCfA | AmsGfsUmAmGmUfUmCmAmU |
| | fUmGmAmAmCmUmAmCmUm | mGfAmUmCfAmAfGmCmCmsAm |
| | _(CR01008×3) | sGm |
| RZ001032 | GmsGmsCmUmUmGmAfUfCfA | AmsGfsUmAmGmUfUmCmAmU |
| | fUmGmAmAmCmUmAmCmUm | mGfAmUmCfA(moe)AfGmCmCm |
| | _(CR01008×3) | sAmsGm |
| RZ001033 | GmsCmsAmGmCmUmCfCfUfUf | UmsAfsUmAmAmCfAmAmUmA |
| | AmUmUmGmUmUmAmUmAm | mAfGmGmAfGmCfUmGmCmsCm |
| | _(CR01008×3) | sAm |
| RZ001034 | GmsCmsAmGmCmUmCfCfUfUf | UmsAfsUmAmAmCfAmAmUmA |
| | AmUmUmGmUmUmAmUmAm | mAfGmGmAfG(moe)CfUmGmCm |
| | _(CR01008×3) | sCmsAm |
| RZ001035 | GmsCmsUmCmCmUmUfAfUfU | UmsCfsGmUmAmUfAmAmCmA |
| | fGmUmUmAmUmAmCmGmAm | mAfUmAmAfGmGfAmGmCmsU |
| | _(CR01008×3) | msGm |
| RZ001038 | CmsUmsGmGmCmUmUfGfAfU | UmsAfsGmUmUmCfUmUmGmA |
| | fCmAmAmGmAmAmCmUmAm | mUfCmAmAfGmCfCmAmGmsCm |
| | _(CR01008×3) | sAm |
| RZ001039 | CmsUmsGmGmCmUmUfGfAfU | UmsAfsGmUmUmCfUmUmGmA |
| | fCmAmAmGmAmAmCmUmAm | mUfCmAmAfG(moe)CfCmAmGm |
| | _(CR01008×3) | sCmsAm |
| RZ001044 | CmsUmsGmAmCmAmCfAfAfU | UmsUfsUmCmUmGfAmGmCmA |
| | fGmCmUmCmAmGmAmAmAm | mUfUmGmUfG(moe)UfCmAmGm |
| | _(CR01008×3) | sAmsUm |

Exemplarily, “_(CR01008×3)” indicates that the ligand represented by (CRO1008×3) is conjugated to the 3′ end of the sense strand.

In some specific embodiments of the present disclosure, the compound conjugated with an oligonucleotide comprises the following group:

| group | sense strand | antisense strand |
|---|---|---|
| | (5′-3′) | (5′-3′) |
| RZ001032 | GmsGmsCmUmUmGmA | AmsGfsUmAmGmUfU |
| | fUfCfAfUmGmAmAm | mCmAmUmGfAmUmCf |
| | CmUmAmCmUm | A(moe)AfGmCmCms |
| | _(CR01008×3) | AmsGm |

### Pharmaceutical Composition

In a second aspect of the present disclosure, provided is a pharmaceutical composition comprising the compound conjugated with an oligonucleotide according to the first aspect of the present disclosure.

In some optional embodiments of the present disclosure, the pharmaceutical composition further comprises one or more pharmaceutically acceptable excipients.

The pharmaceutical composition of the present disclosure includes formulations suitable for parenteral administration. The formulations may be conveniently presented in unit dose forms and may be prepared by any method known in the field of pharmacy. The amount of active ingredient that can be combined with an excipient substance to prepare a single dose form is generally the amount of siRNA that produces a therapeutic effect.

### Use

In a third aspect of the present disclosure, provided is use of the compound conjugated with an oligonucleotide according to the first aspect of the present disclosure or the pharmaceutical composition according to the second aspect of the present disclosure in the manufacture of a medicament for alleviating, preventing and/or treating the LPA gene-mediated disease or condition.

In some optional embodiments of the present disclosure, the LPA gene-mediated disease or condition includes cardiovascular disease, the cardiovascular disease includes but is not limited to hyperlipoproteinemia(a), Berger's disease, peripheral artery disease, coronary artery disease, metabolic syndrome, acute coronary syndrome, aortic stenosis, aortic regurgitation, aortic dissection, cerebrovascular disease, mesenteric ischemia, superior mesenteric artery occlusion, renal artery stenosis, stable/unstable angina pectoris, acute coronary syndrome, heterozygous or homozygous familial hypercholesterolemia, high apolipoprotein beta lipoprotein, cerebrovascular atherosclerosis, cerebrovascular disease and venous thrombosis, stroke, atherosclerosis, thrombosis, coronary artery disease and/or any other disease or condition related to the increased level of LP(a) particles.

### Kit

In a fourth aspect of the present disclosure, provided is a kit comprising the compound conjugated with an oligonucleotide according to the first aspect of the present disclosure or the pharmaceutical composition according to the second aspect of the present disclosure.

### Method for Inhibiting LPA Gene Expression

In a fifth aspect of the present disclosure, provided is a method for inhibiting LPA gene expression in a subject in need thereof, wherein the method comprises administering to the subject the compound conjugated with an oligonucleotide according to the first aspect of the present disclosure or the pharmaceutical composition according to the second aspect of the present disclosure.

In some specific embodiments of the present disclosure, a method for inhibiting LPA expression in a cell in vitro comprises contacting the cell with the compound conjugated with an oligonucleotide according to the first aspect of the present disclosure or the pharmaceutical composition according to the second aspect of the present disclosure.

In some optional embodiments of the present disclosure, the inhibiting the expression of LPA gene in a cell is performed for a period of time that is sufficient to degrade the mRNA transcript of the LPA gene.

### Method for Treating a Disease

In a sixth aspect of the present disclosure, provided is a method for alleviating, treating and/or preventing the LPA-mediated disease or condition in a subject in need thereof, wherein the method comprises administering to the subject the compound conjugated with an oligonucleotide according to the first aspect of the present disclosure or the pharmaceutical composition according to the second aspect of the present disclosure.

In some specific embodiments of the present disclosure, the subject is a human.

In some specific embodiments of the present disclosure, the administration includes subcutaneous administration or intravenous administration.

In some specific embodiments of the present disclosure, the LPA gene-mediated disease or condition includes a disease related to mRNA expression level of LPA gene.

In some specific embodiments of the present disclosure, the LPA gene-mediated disease or condition includes cardiovascular disease, the cardiovascular disease includes but is not limited to hyperlipoproteinemia(a), Berger's disease, peripheral artery disease, coronary artery disease, metabolic syndrome, acute coronary syndrome, aortic stenosis, aortic regurgitation, aortic dissection, cerebrovascular disease, mesenteric ischemia, superior mesenteric artery occlusion, renal artery stenosis, stable/unstable angina pectoris, acute coronary syndrome, heterozygous or homozygous familial hypercholesterolemia, high apolipoprotein beta lipoprotein, cerebrovascular atherosclerosis, cerebrovascular disease and venous thrombosis, stroke, atherosclerosis, thrombosis, coronary artery disease and/or any other disease or condition related to the increased level of LP(a) particles.

The compound conjugated with an oligonucleotide and the pharmaceutical composition thereof show excellent LPA gene expression modulating activity in mouse model experiments. For example, the compound conjugated with an oligonucleotide and the pharmaceutical composition thereof provided by the present disclosure can significantly inhibit expression of LPA mRNA in animal liver tissue, and the effect level is better than that of Olpasiran. Therefore, the compound conjugated with an oligonucleotide and the pharmaceutical composition thereof provided by the present disclosure are useful for regulating the expression level of the target gene LPA, alleviating, preventing and/or treating a disease or condition mediated by the mRNA level of LPA gene expression, with good application prospects.

To make the purposes, technical solutions, and advantages of the present disclosure clearer, embodiments of the present disclosure are described in detail below in conjunction with examples.

Unless otherwise indicated, the reagents used in the production of the compounds of the present disclosure were purchased from Beijing Ouhe Technology Co., LTD., wherein the information of the main reagents is shown in Table 1.

TABLE 1
Main reagents

| Reagent | Abbreviation | CAS number |
|---|---|---|
| Lithium aluminum hydride (LiAlH4) | — | 16853-85-3 |
| Wet palladium carbon (10 mass % loading) | Pd/C | — |
| Palladium carbon hydroxide (10 mass % loading) | Pd(OH)2/C | — |
| Benzotriazole-N,N,N′,N′-tetramethylurea | HBTU | 94790-37-1 |
| hexafluorophosphate | | |
| 2-(7-azobenzotriazole)-N,N,N′,N′- | HATU | 148893-10-1 |
| tetramethylurea hexafluorophosphate | | |
| 4,4′-Bismethoxy triphenylmethyl chloride/4,4′- | DMTrCl | 40615-36-9 |
| dimethoxy triphenyl chloromethane | | |
| Bis(diisopropylamino) (2-cyanoethoxy)phosphine | — | 102691-36-1 |
| 4,5-Dicyanoimidazole | DCl | 1122-28-7 |
| 4-Dimethylaminopyridine | DMAP | 1122-58-3 |
| Aminoalkyl-CPG, Model: C3006-1000 | 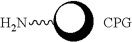 | — |
| 4M hydrochloric acid in 1,4-dioxane solution | — | — |
| Trans-4-(Boc-amino)cyclohexyl formaldehyde | — | 181308-57-6 |
| N-benzyloxycarbonyl-4-aminobutyric acid | — | 5105-78-2 |
| 5-[[(2R,3R,4R,5R,6R)-3-acetamido-4,5- | Compound 4 | 1159408-54-4 |
| diacetoxy-6-(acetoxymethyl)-2- | | |
| tetrahydropyranyl]oxy]valeric acid | | |

Wherein, CPG represents controlled pore glass support.

Unless otherwise indicated, the reagents, consumables and instruments used in the biological assay of the present disclosure are commercially available. Among them, the main reagents and consumables are detailed in Table 2, and the main instruments are detailed in Table 3.

TABLE 2
Main reagents and consumables

| Reagent | Manufacturer |
|---|---|
| 1 × PBS | M&C GENE |
| | TECHNOLOGY(BEIJING) |
| | LTD. |
| DMEM Medium | M&C GENE |
| | TECHNOLOGY(BEIJING) |
| | LTD. |
| Opti-MEM ™ Medium | Gibco |
| Fetal bovine serum | Sigma |
| Trypsin | M&C GENE |
| | TECHNOLOGY(BEIJING) |
| | LTD. |
| Double antibiotics | BBI |
| Lipofectamine RNAiMax | Invitrogen |
| Nucleic acid extraction | Zhejiang Hanwei |
| or purification kit | Technology Co., Ltd. |
| RevertAid First Strand | Thermo Fisher Scientific |
| cDNA Synthesis Kit | |
| TaqMan Fast Advanced Master Mix | Thermo Fisher Scientific |
| RNA Extraction Kit (HanWei) | Zhejiang Hanwei |
| | Technology Co., Ltd. |
| RNALater | Thermo Fisher Scientific |
| human Lipoprotein A ELISA kit | Abcam |
| ZoIetil 50 | French Vike Co., Ltd. |

TABLE 3
Main instruments

| Instrument | Manufacturer |
|---|---|
| Fully automatic nucleic acid | Zhejiang Hanwei |
| extraction equipment | Technology Co., Ltd. |
| High-speed freezing centrifuge | Eppendorf |
| Carbon dioxide incubator | Thermo Fisher Scientific |
| Biological safety cabinet | Shanghai Lishen |
| Constant temperature water bath pot | Shanghai Boxun |
| Automatic cell counter | Shanghai Countstar |
| Inverted microscope | Olympus |
| NANODROP OneC | Thermo Fisher Scientific |
| Gradient PCR Amplifier | Eppendorf |
| CFX Opus 384 | Bio-Rad |
| LightCycler 480 | Roche |
| Paraffin slicer | Jinhua YIDI |
| Tissue Dehydrator | Leica |
| Fully automatic biological | Jinhua YIDI |
| tissue embedding machine | |
| Automatic hematology analyzer | SYSMEX |
| Automatic biochemical analyzer | SYSMEX |
| Automated coagulation analyzer | Mindray |

The reagent ratios described in the present disclosure are calculated as volume-to-volume (v/v), unless otherwise noted.

### Preparation of Compounds

### Preparative Example 1: Synthesis of Compounds CR01008 and CR01008Z

### (1.1) Synthesis of Compound CR01008

In this preparative example, the synthetic route of compound CR01008 is shown below.

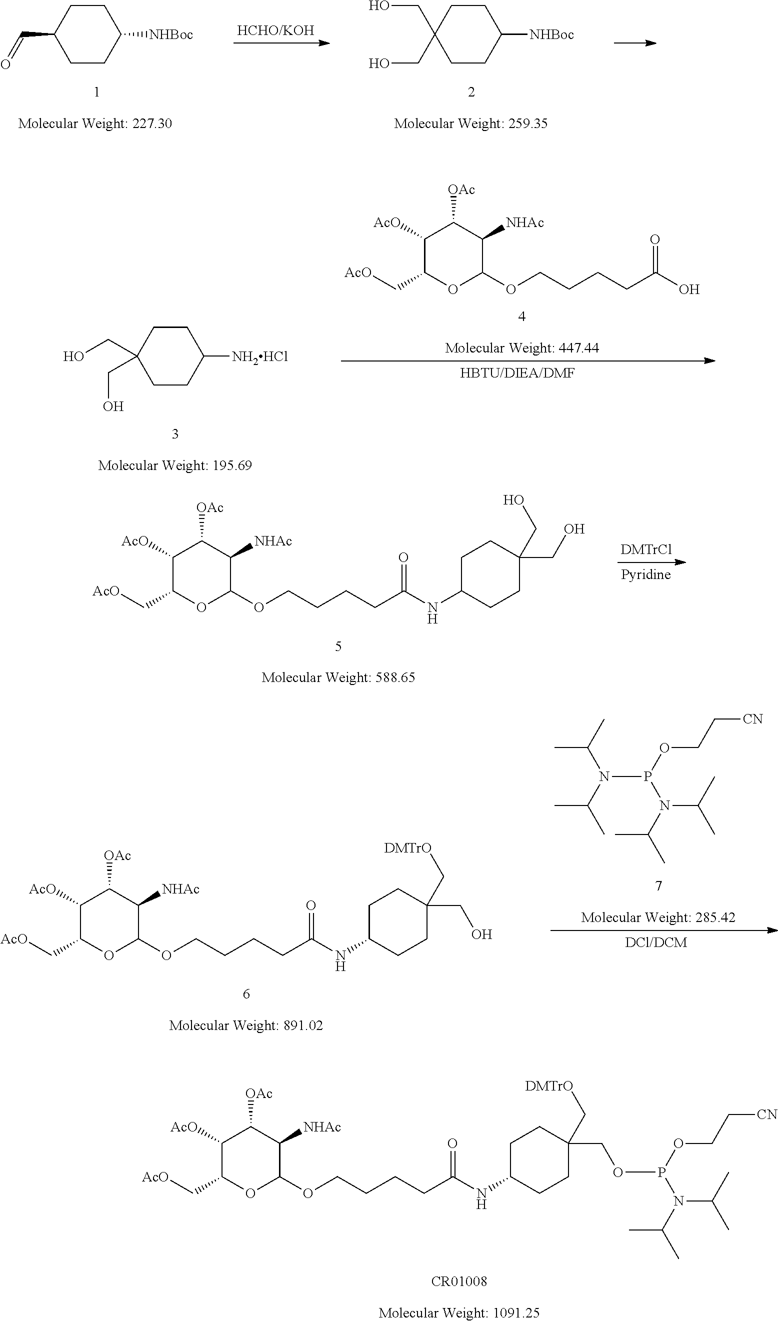

### (1.1.1) Synthesis of Compound 2

Compound 1 (trans-4-(Boc-amino)cyclohexylformaldehyde, 10.0 g, 1.0 eq) and aqueous formaldehyde solution (8.9 g, 37 mass %, 2.4 eq) were dissolved in 33 ml of methanol, and 13 ml of an aqueous KOH solution at a concentration of 45.3 mass % was added dropwise. After the dropwise addition was completed, the reaction system was stirred for 30 min at 25° C., heated up to 60° C., and refluxed at 60° C. for 2 h of reaction. After the reaction was completed, the reaction solution was cooled to room temperature and evaporated to dryness under reduced pressure to obtain a crude product as white solid. The crude product was slurried with a small amount of water, and then filtered to obtain compound 2 as a white solid (9 g, 78.9% yield). MS-ESI (m/z)=260 [M+H]⁺.

### (1.1.2) Synthesis of Compound 3

The compound 2 (9 g, 1 eq) prepared according to step (1.1.1) was dissolved in 70 ml of 1,4-dioxane, added with a solution of hydrogen chloride in 1,4-dioxane (45 ml, 4 M) and stirred at 25° C. for 1 h of reaction. After the reaction was completed, the reaction solution was evaporated to dryness under reduced pressure to obtain compound 3 as a white solid (6.8 g, 100% yield).

### (1.1.3) Synthesis of Compound 5

The compound 3 (1.8 g, 2.0 eq) prepared according to step (1.1.2), compound 4 (5-[[(2R,3R,4R,5R,6R)-3-acetamido-4,5-diacetoxy-6-(acetyloxymethyl)-2-tetrahydropyranyl]oxy]pentanoic acid, 2.1 g, 1.0 eq), and DIEA (N,N-diisopropylethylamine, 3.5 g, 6.0 eq) were dissolved in 15 ml of DMF, added with HBTU (1.9 g, 1.1 eq), and stirred at 25° C. under N₂ atmosphere for 3 h of reaction. After the reaction was completed, the reaction solution was evaporated to dryness under reduced pressure and purified by reverse phase chromatography (22 vol % aqueous solution of acetonitrile) to obtain compound 5 as a white solid (1.78 g, 64.4% yield). MS-ESI (m/z)=589[M+H]⁺.

### (1.1.4) Synthesis of Compound 6

The compound 5 (1.54 g, 1.0 eq) prepared according to step (1.1.3) was dissolved in 15 ml of pyridine, the reaction system was cooled down to 0° C. in an ice-water bath and DMTrCl (4,4′-dimethoxytriphenylmethyl chloride, 1.32 g, 1.5 eq) was added at 0° C. to perform 3 h of reaction at 25° C. The reaction was quenched by adding 15 ml of methanol to the reaction solution. After the reaction was completed, the reaction solution was evaporated to dryness under reduced pressure and purified by reverse phase chromatography (60 vol % aqueous solution of acetonitrile) to obtain compound 6 as a yellow solid (1 g, 42.7% yield). MS-ESI (m/z)=891 [M+H]⁺.

### (1.1.5) Synthesis of Compound CR01008

The compound 6 (1.08 g, 1.0 eq) prepared according to step (1.1.4) was dissolved in 20 ml of anhydrous dichloromethane, DCI (115 mg, 0.8 eq) and compound 7 (bis(diisopropylamino)(2-cyanoethoxy)phosphine, 732 mg, 2.1 eq) were added separately, nitrogen replacement was performed three times, and the reaction system was stirred at 25° C. for 2 h of reaction. After the reaction was completed, the reaction solution was added with 20 ml of saturated sodium bicarbonate aqueous solution and extracted with 20 ml of dichloromethane 3 times (3×20 ml). The organic phases were combined, evaporated to dryness under reduced pressure, purified by reverse phase chromatography (72 vol % aqueous solution of acetonitrile) and then dried under vacuum for 12 h to obtain the compound CR01008 as a white powder (1 g, 76.0% yield). MS-ESI (m/z)=1091 [M+Na]⁺.

1H NMR (400 MHz, DMSO-d6) δ 1.05 (d, J=6.7 Hz, 6H). 1.14 (d, J=6.7 Hz, 6H), 1.37-1.17 (m, 5H), 1.60-1.40 (m, 6H), 1.68-1.62 (m, 1H), 1.80 (s, 3H), 1.80 (s, 3H), 1.92 (s, 3H), 2.02 (s, 5H), 2.13 (s, 3H), 2.71 (t, J=5.9 Hz, 2H), 2.79 (d, J=8.4 Hz, 1H), 2.87 (d, J=8.4 Hz, 1H), 3.36 (s, 1H), 3.58-3.39 (m, 3H), 3.69-3.60 (m, 2H), 3.75 (s, 7H), 3.90 (dt, J=11.2, 8.8 Hz, 1H), 4.05 (s, 3H), 4.51 (d, J=8.4 Hz, 1H), 4.99 (dd, J=11.3, 3.4 Hz, 1H), 5.24 (d, J=3.4 Hz, 1H), 5.78 (s, 1H), 6.93-6.87 (m, 4H), 7.35-7.21 (m, 7H), 7.44-7.37 (m, 2H), 7.66 (d, J=7.8 Hz, 1H), 7.84 (d, J=9.2 Hz, 1H).

### (1.2) Synthesis of Compound CR01008Z

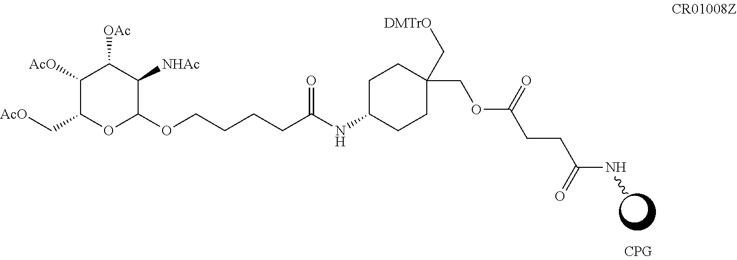

In this example, the synthetic route of compound CR01008Z is shown below.

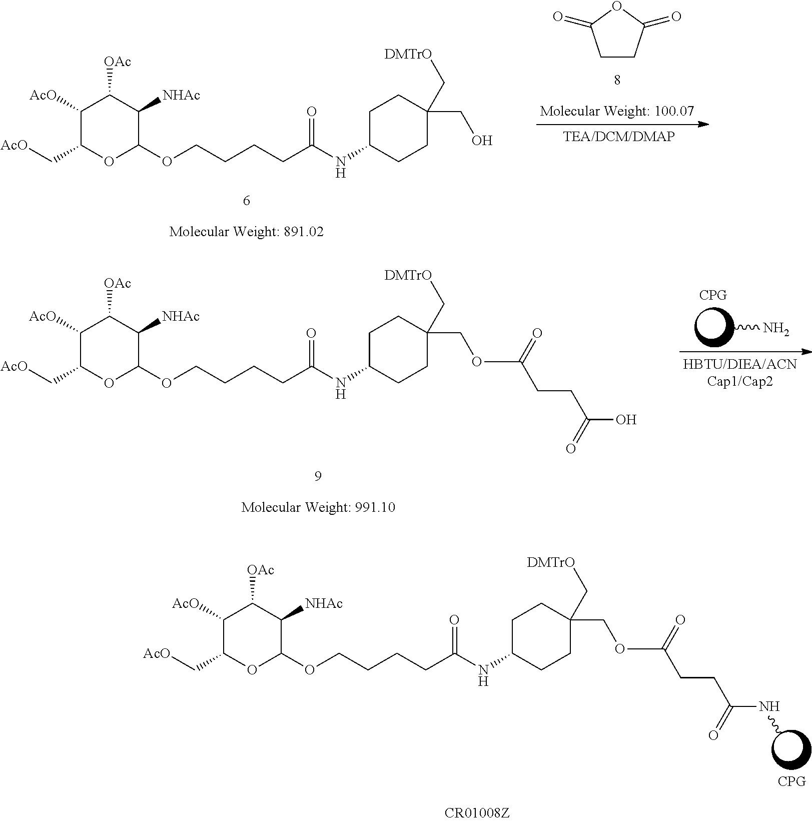

### (1.2.1) Synthesis of Compound 9

The compound 6 prepared according to step (1.1.4) (500 mg) was dissolved in 10 ml of dichloromethane, compound 8 (succinic anhydride, 112 mg), DMAP (6.8 mg) and TEA (226.2 mg) were added, nitrogen replacement was performed three times, and the reaction system was stirred at 25° C. for 16 h of reaction. The reaction solution was purified by flash chromatography to obtain compound 9 (300 mg, 53.6% yield). MS-ESI (m/z)=1013 [M+Na]⁺.

### (1.2.2) Synthesis of Compound CR01008Z

The compound 9 (50 mg) prepared according to step (1.2.1), aminoalkyl CPG (1.25 g, 80 mol/g, 0.1 mmol), HBTU (27 mg), and DIEA (12 mg) were added to a 20 ml vial, and the reaction was carried out on a shaker for 16 h. After the reaction was completed, the reaction solution was filtered to obtain a filter cake. The filter cake was washed once with 10 ml of acetonitrile (1×10 ml) and then dried under vacuum. The dried filter cake, DMAP (3 mg), Cap1 (10 ml, 200 V) and Cap2 (1 ml, 20 V) were added to a 20 ml vial, and the reaction was carried out on a shaker for 6 h. After the reaction was completed, the reaction solution was filtered to obtain a filter cake. The filter cake was washed once with 10 ml of acetonitrile (1×10 ml) and then dried under vacuum to obtain the compound CR01008Z (1.03 g, loading amount: 20-30 mol/g).

Wherein, Cap1 and Cap2 were capping agents, Cap1 was a solution of 20 vol % N-methylimidazole in a pyridine/acetonitrile mixture, the volume ratio of pyridine to acetonitrile was 3:5, and Cap2 was a solution of 20 vol % acetic anhydride in acetonitrile.

### Preparative Example 2: Preparation of Compounds CR01013 and CR01013Z

### (2.1) Preparation of Compound CR01013

In this example, the synthetic route of compound CR01013 is shown below.

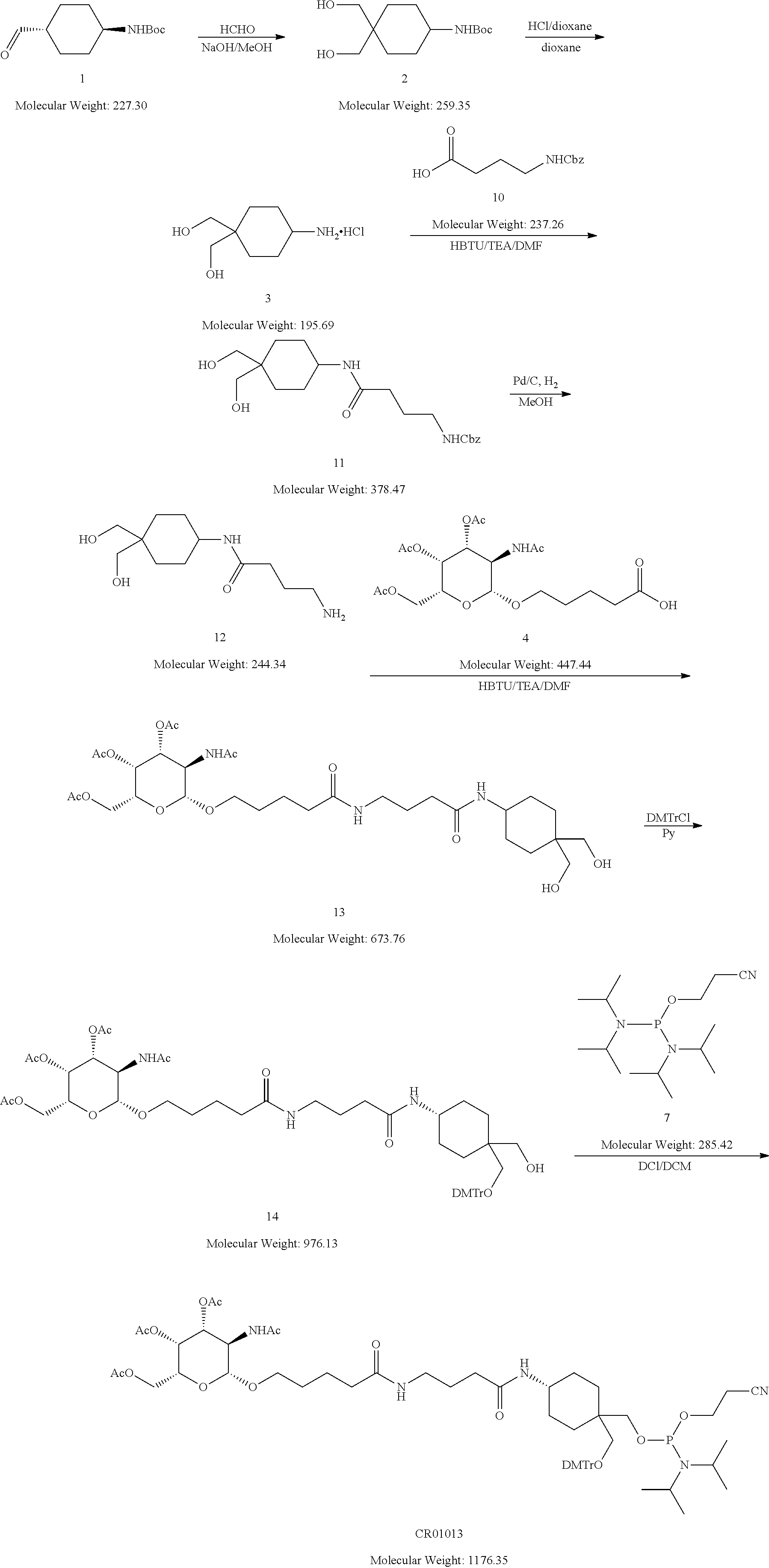

### (2.1.1) Synthesis of Compound 2

Compound 1 (trans-4-(Boc-amino)cyclohexylcarboxaldehyde, 4.9 g) was dissolved in 17 ml of methanol. An aqueous solution of formaldehyde (4.21 g, at a concentration of 37 mass %) and an aqueous solution of sodium hydroxide (6.5 ml, at a concentration of 45.3 mass %) were added dropwise. After the dropwise addition was completed, the reaction system was heated to 60° C., and stirred at 60° C. for 2 h of reaction. After the reaction was completed, the reaction solution was cooled to 25° C. and evaporated to dryness under reduced pressure to obtain a crude product as a white solid. The crude product was slurried with a small amount of water, filtered and dried to obtain compound 2 as a white solid (4.8 g, 85.9% yield). ESI-MS (m/z)=260.2[M+H]⁺.

### (2.1.2) Synthesis of Compound 3

The compound 2 (4.8 g) prepared according to step (2.1.1) was dissolved in 25 ml of 1,4-dioxane, a solution of hydrochloric acid in 1,4-dioxane (25 ml, 4 M) was added, and the reaction system was stirred and reacted at 25° C. for 2 h. After the reaction was completed, the reaction solution was evaporated to dryness under reduced pressure to obtain compound 3 as a white solid (3.6 g, 99.4% yield).

### (2.1.3) Synthesis of Compound 11

The compound 3 (3.6 g) prepared according to step (2.1.2) was dissolved in 36 ml of DMF, and then TEA (5.62 g), compound 10 (N-benzyloxycarbonyl-4-aminobutyric acid, 5.28 g), and HBTU (8.43 g) were added, and the reaction system was stirred and reacted at 25° C. for 16 h. After the reaction was completed, 200 ml of saturated aqueous sodium bicarbonate solution was added. The reaction solution was extracted with 100 ml of ethyl acetate three times (3×100 ml), the organic phases were combined, washed with 50 ml of saturated aqueous solution of sodium chloride (1×50 ml), dried over anhydrous sodium sulfate, evaporated to dryness under reduced pressure, and then purified by normal-phase chromatography (eluent: dichloromethane/methanol=10/1, v/v) to obtain compound 11 as a white solid (2.3 g, 33.0% yield). ESI MS (m/z)=379.5[M+H]⁺.

### (2.1.4) Synthesis of Compound 12

The compound 11 (2.3 g) prepared in step (2.1.3) was dissolved in 23 ml of methanol, wet palladium carbon (230 mg, 10 mass % loading) was added, hydrogen replacement was performed three times, and the reaction system was stirred and reacted at 25° C. for 16 h under a hydrogen atmosphere (15 psi). After the reaction was completed, the reaction solution was filtered to obtain a filtrate, and the filtrate was evaporated to dryness under reduced pressure to obtain compound 12 as a yellow oil (1.48 g, 99.8% yield).

### (2.1.5) Synthesis of Compound 13

The compound 12 (1.48 g) prepared in step (2.1.4) was dissolved in 15 ml of DMF, triethylamine (TEA, 1.22 g), compound 4 (1.35 g), and HBTU (3.45 g) were added, and the reaction system was stirred and reacted at 25° C. for 16 h. After the reaction was completed, 150 ml of saturated aqueous solution of sodium bicarbonate was added, the reaction solution was extracted with 50 ml of ethyl acetate for three times (3×50 ml). The organic phases were combined, washed with 30 ml of saturated aqueous solution of sodium chloride (1×30 ml), dried over anhydrous sodium sulfate, evaporated to dryness under reduced pressure, and then purified by reverse phase chromatography (C18 column, eluent: water/acetonitrile=5/1, v/v) to obtain compound 13 as a white solid (1.3 g, 31.8% yield). ESI-MS (m/z): 674.3 [M+H]⁺.

### (2.1.6) Synthesis of Compound 14

The compound 13 (1.1 g) prepared in step (2.1.5) was dissolved in 11 ml of pyridine, the reaction system was cooled down to 0° C. in an ice-water bath, and DMTrCl (813 mg) was added in batches at 0° C. The reaction system was stirred and reacted at 0° C. for 1 h. After the reaction was completed, the reaction solution was added with methanol to quench the reaction, evaporated to remove solvent, and purified by reverse-phase chromatography (eluent: water/acetonitrile=1/4, v/v) to obtain compound 14 as a white solid (800 mg, 50.3% yield). ESI-MS (m/z):976.5[M+H]⁺.

### (2.1.7) Synthesis of Compound CR01013

At 25° C., the compound 14 (550 mg) prepared in step (2.1.6) was dissolved in 5 ml of dichloromethane (DCM), and 4,5-dicyanoimidazole (DCl, 53.2 mg) and compound 7 (2-cyanoethyl N,N,N′,N′-tetraisopropyl phosphordiamidite, 255.4 mg) were added. Nitrogen replacement was performed three times, and the reaction system was stirred at 25° C. in nitrogen atmosphere for 1 h. After the reaction was completed, the reaction solution was washed twice with 5 ml of saturated aqueous sodium bicarbonate solution (2×5 ml) and then once with 30 ml of saturated aqueous sodium chloride solution (1×30 ml). The organic phase was separated, dried over anhydrous sodium sulfate, evaporated under reduced pressure to remove solvent, and purified by normal-phase chromatography (eluent: dichloromethane/methanol=20/1, v/v) to obtain compound CR01013 as a white solid (532 mg, 80.4% yield). ESI-MS (m/z): 1176.7[M+H]⁺.

1H NMR (400 MHz, DMSO-d6) δ 0.95-1.05 (d, J=6.7 Hz, 5H), 1.06-1.15 (q, J=7.6 Hz, 8H), 1.15-1.21 (t, J=7.2 Hz, 14H), 1.72-1.80 (s, 3H), 1.84-1.92 (s, 3H), 1.94-2.07 (d, J=16.0 Hz, 7H), 2.07-2.14 (s, 3H), 2.64-2.72 (q, J=5.8 Hz, 2H), 2.74-2.89 (d, J=8.5 Hz, 2H), 3.35-3.56 (m, 4H), 3.57-3.70 (m, 4H), 3.71-3.77 (s, 6H), 3.81-3.93 (m, 1H), 3.96-4.09 (d, J=6.4 Hz, 3H), 6.82-6.97 (d, J=8.7 Hz, 4H), 7.17-7.27 (t, J=8.7 Hz, 5H), 7.27-7.34 (t, J=7.6 Hz, 2H), 7.34-7.43 (d, J=7.5 Hz, 2H).

### (2.2) Synthesis of Compound CR01013Z

In this example, the synthetic route of compound CR01013Z is shown below.

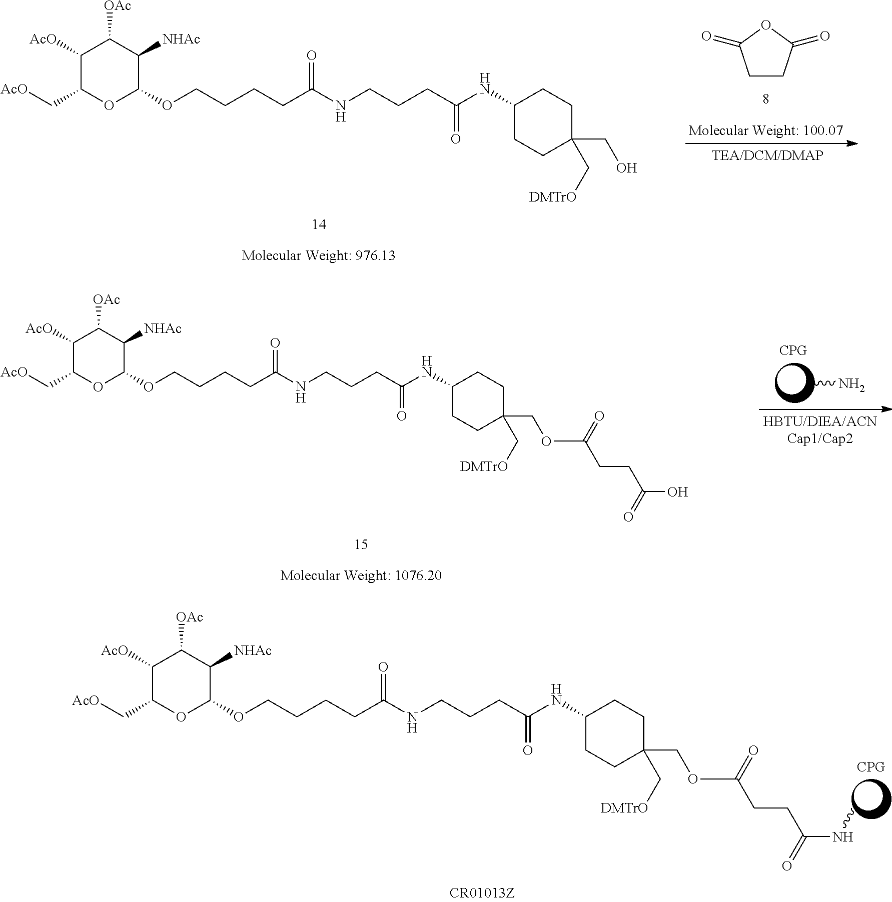

### (2.2.1) Synthesis of Compound 15

At 25° C., the compound 14 (100 mg, 0.10 mmol) prepared in step (2.1.6) was dissolved in 2 ml of dichloromethane, and triethylamine (25.9 mg, 0.25 mmol), DMAP (1.25 mg, 0.01 mmol) and compound 8 (succinic anhydride, 15.4 mg, 0.15 mmol) were added. The reaction system was stirred at 25° C. for 16 h. After the reaction was completed, the reaction solution was evaporated to remove solvent and purified by reverse-phase chromatography (C18 column, eluent: water/acetonitrile=2/1, v/v) to obtain compound 15 as ayellow oil (110 mg, 0.10 mmol, 100% yield). ESI-MS (m/z)=1099.3 [M+Na]⁺.

### (2.2.2) Synthesis of Compound CR01013Z

The compound 15 (50 mg, 0.04 mmol) prepared in step (2.2.1) was dissolved in 10 ml of acetonitrile, HBTU (24.2 mg, 0.06 mmol), DIEA (11.0 mg, 0.08 mmol) and aminoalkyl-CPG (1.06 g, loading amount: 80 mol/g) were added, and the reaction system was stirred at 25° C. for 16 h of reaction. After the reaction was completed, the reaction solution was filtered to obtain a filter cake, and the filter cake was washed twice with 50 ml of dichloromethane (2×50 ml), three times with 50 ml of acetonitrile (3×50 ml), and once with 50 ml of ethyl acetate (1×50 ml) in sequence, and then dried under vacuum. Cap1 (4.8 ml), Cap2 (0.54 ml) and DMAP (2.59 mg) were added to the dried filter cake, and the reaction system was stirred and reacted at 25° C. for 5 h. After the reaction was completed, the reaction solution was filtered to obtain a filter cake. The filter cake was washed three times with 50 ml of acetonitrile (3×50 ml) and dried under vacuum to obtain compound CR01013Z (900 mg, loading amount: 20-30 mol/g).

Wherein, Cap1 and Cap2 were capping agents, Cap1 was a solution of 20 vol % N-methylimidazole in a pyridine/acetonitrile mixture, the volume ratio of pyridine to acetonitrile was 3:5, and Cap2 was a solution of 20 vol % acetic anhydride in acetonitrile.

### Preparative Example 3: Preparation of Double-Stranded Oligonucleotide (SiRNA)

### (3.1) Synthesis of Sense Strand (SS)

According to the solid-phase nucleic acids synthesis using phosphoramidite method, nucleoside monomers were linked one by one in the direction from 3′ to 5′. The linking of each nucleoside monomer comprised a four-step reaction of deprotection, coupling, capping, and oxidation or sulfurization. The synthesis condition was given below.

The nucleoside monomers were prepared to a solution of 0.1 M nucleoside monomer in acetonitrile.

The condition for deprotection reaction in each step was identical, including a temperature of 25° C., a reaction time of 70 seconds, a solution of dichloroacetic acid in dichloromethane (3 vol %) as a deprotection agent, and a molar ratio of the dichloroacetic acid to the protecting group 4,4′-dimethoxytrityl on the solid phase support of 5:1.

The condition for coupling reaction in each step was identical, including a temperature of 25° C., a molar ratio of the nucleic acid sequence linked to the solid phase support to the nucleoside monomers of 1:10, a molar ratio of the nucleic acid sequence linked to the solid phase support to a coupling agent of 1:65, a reaction time of 600 seconds, a solution of 0.5 M 5-ethylthio-1H-tetrazole in acetonitrile as a coupling agent, and a solution of 0.2 M xanthane hydride in a acetonitrile/pyridine mixture (volume ratio of acetonitrile:pyridine=1:1) as a thio agent.

The condition for capping reaction in each step was identical, including a temperature of 25° C., a reaction time of 2 min, a mixed solution of Cap1 and Cap2 in a molar ratio of 1:1 as a capping agent, a solution of 20 vol % N-methylimidazole in a pyridine/acetonitrile mixture as Cap1, a volume ratio of pyridine to acetonitrile of 3:5, a solution of 20 vol % acetic anhydride in acetonitrile as Cap2, and a molar ratio of N-methylimidazole in the capping agent Cap1:acetic anhydride in the capping agent Cap2: the nucleic acid sequence connected to the solid phase support of 1:1:1.

The condition for oxidation reaction in each step was identical. The condition for oxidation reaction included a temperature of 25° C., a reaction time of 3 seconds, 0.05 M iodine water as an oxidation agent, and a molar ratio of iodine to the nucleic acid sequence connected to the solid phase support in the coupling reaction of 30:1. The oxidation reaction was carried out in a mixed solvent of water/pyridine (volume ratio of water:pyridine=1:9). The condition for sulfurization reaction included a temperature of 25° C., a reaction time of 360 seconds, a solution of 0.2 M xanthane hydride in pyridine as a thio agent, and a molar ratio of the thio agent to the nucleic acid sequence connected to the solid phase support in the coupling reaction of 4:1. The sulfurization reaction was carried out in a mixed solvent of water/pyridine (volume ratio of water:pyridine =1:9).

After the last nucleoside monomer was linked, the nucleic acid sequence connected to the solid phase support was cleaved, deprotected, purified and desalted in turn, and then freeze-dried to obtain the sense strand.

The conditions for cleavage and deprotection were as follows: the synthesized nucleotide sequence connected to the solid phase support was added into a 25 mass % aqueous ammonia solution for 16 h of reaction at 55° C., wherein the aqueous ammonia solution was used in an amount of 0.5 ml/mol. The solvent was removed, and the residue was concentrated under vacuum to dryness. After the treatment by aqueous ammonia solution was completed, the resulting product was dissolved with 0.4 ml/mol N-methylpyrrolidone according to the amount of single-stranded nucleic acid, and then added with 0.3 ml/mol triethylamine and 0.6 ml/mol triethylamine trihydrofluoride to remove 2′-O-TBDMS protection from the ribose.

The conditions for purification and desalination were as follows: the nucleic acids were purified using a preparative ion chromatography column (Source 15Q) with a gradient elution by NaCl. Specifically, eluent 1 was 20 mM sodium phosphate (pH 8.1) in a mixed solvent of water/acetonitrile (volume ratio of water:acetonitrile=9:1); eluent 2 was 1.5 M sodium chloride and 20 mM sodium phosphate (pH 8.1) in a mixed solvent of water/acetonitrile (volume ratio of water:acetonitrile=9:1); elution gradient was eluent 1:eluent 2=(100:0) to (50:50). The eluate was collected, combined and desalted by using a reverse phase chromatography purification column. The conditions for desalination included a sephadex column (packing material: Sephadex-G25) for desalination, and deionized water for elution.

Detection: The purity detection was performed using ion exchange chromatography (IEX-HPLC), and the molecular weight was measured by liquid chromatography-mass spectrometry (LC-MS). The measured value of the molecular weight and the theoretical value of the molecular weight were compared. When the measured value was consistent with the theoretical value, it indicated that a sense strand of siRNA was obtained.

### (3.2) Synthesis of Antisense Strand (AS)

Antisense strands were synthesized using a general solid phase support. The reaction conditions of deprotection, coupling, capping, oxidation or sulfurization, cleavage and deprotection, and purification and desalination in the solid-phase synthesis method of antisense strand were the same as those used for the synthesis of the sense strand in step (3.1).

Detection: The purity detection was performed using ion exchange chromatography (IEX-HPLC), and the molecular weight was measured by liquid chromatography-mass spectrometry (LC-MS). The measured value of the molecular weight and the theoretical value of the molecular weight were compared. When the measured value was consistent with the theoretical value, it indicated that an antisense strand of siRNA was obtained.

(3) Synthesis of Double-Stranded siRNA

The sense strand synthesized in step (3.1) and the antisense strand synthesized in step (3.2) were mixed at an equimolar ratio, dissolved in water for injection, heated to 95° C., slowly cooled to room temperature and left to stand at room temperature for 10 min to allow the sense and antisense strands to form a double-stranded structures by hydrogen bonds, thereby obtaining the siRNA shown in Table 5.

Unmodified double-stranded oligonucleotides (siRNAs) prepared according to the methods provided in the present disclosure are shown in Table 4.

TABLE 4
Unmodified siRNA

| Number | | | | |
|---|---|---|---|---|
| of | Sense strand | | Antisense | |
| siRNA | (5′-3′) | SEQ ID | strand (5′-3′) | SEQ ID |
| 1 | GGCUUGAUCA | No. 1 | AGUAGUUCAUG | No. 2 |
| | UGAACUACU | | AUCAAGCCAG | |
| 2 | GCAGCUCCUU | No. 3 | UAUAACAAUAA | No. 4 |
| | AUUGUUAUA | | GGAGCUGCCA | |
| 3 | GCUCCUUAUU | No. 5 | UCGUAUAACAA | No. 6 |
| | GUUAUACGA | | UAAGGAGCUG | |
| 4 | CGGUAAUGGA | No. 7 | AUAACUCUGUC | No. 8 |
| | CAGAGUUAU | | CAUUACCGUG | |
| 5 | CUGGCUUGAU | No. 9 | UAGUUCUUGAU | No. 10 |
| | CAAGAACUA | | CAAGCCAGCA | |
| 6 | AGAGGACAAC | No. 11 | UAAUAUUCUGU | No. 12 |
| | AGAAUAUUA | | UGUCCUCUGA | |
| 7 | CUGACACAAU | No. 13 | UUUCUGAGCAU | No. 14 |
| | GCUCAGAAA | | UGUGUCAGAU | |

The sequence listing software requires “U” to be represented by “T” when editing RNA sequences, so the sequence list does not correctly characterize the siRNA sequences of the present disclosure, and all sequences are subjected to the specification.

The modified double-stranded oligonucleotides (siRNAs) shown in Table 5 were prepared by methods provided by the present disclosure.

TABLE 5
Modified siRNA sequences

| Group | Sense strand (5′-3′) | Antisense strand (5′-3′) |
|---|---|---|
| 1 | GmsGmsCmUmUmGmAfUfCfAf | AmsGfsUmAmGmUfUmCmAmUmGf |
| | UmGmAmAmCmUmAmCmUm | AmUmCfAmAfGmCmCmsAmsGm |
| 2 | GmsGmsCmUmUmGmAfUfCfAf | AmsGfsUmAmGmUfUmCmAmUmGf |
| | UmGmAmAmCmUmAmCmUm | AmUmCfA(moe)AfGmCmCmsAmsGm |
| 3 | GmsCmsAmGmCmUmCfCfUfUf | UmsAfsUmAmAmCfAmAmUmAmAf |
| | AmUmUmGmUmUmAmUmAm | GmGmAfGmCfUmGmCmsCmsAm |
| 4 | GmsCmsAmGmCmUmCfCfUfUf | UmsAfsUmAmAmCfAmAmUmAmAf |
| | AmUmUmGmUmUmAmUmAm | GmGmAfG(moe)CfUmGmCmsCmsAm |
| 5 | GmsCmsUmCmCmUmUfAfUfUf | UmsCfsGmUmAmUfAmAmCmAmAf |
| | GmUmUmAmUmAmCmGmAm | UmAmAfGmGfAmGmCmsUmsGm |
| 6 | CmsGmsGmUmAmAmUfGfGfAf | AmsUfsAmAmCmUfCmUmGmUmCfC |
| | CmAmGmAmGmUmUmAmUm | mAmUfUmAfCmCmGmsUmsGm |
| 7 | CmsGmsGmUmAmAmUfGfGfAf | AmsUfsAmAmCmUfCmUmGmUmCfC |
| | CmAmGmAmGmUmUmAmUm | mAmUfT(moe)AfCmCmGmsUmsGm |
| 8 | CmsUmsGmGmCmUmUfGfAfUf | UmsAfsGmUmUmCfUmUmGmAmUf |
| | CmAmAmGmAmAmCmUmAm | CmAmAfGmCfCmAmGmsCmsAm |
| 9 | CmsUmsGmGmCmUmUfGfAfUf | UmsAfsGmUmUmCfUmUmGmAmUf |
| | CmAmAmGmAmAmCmUmAm | CmAmAfG(moe)CfCmAmGmsCmsAm |
| 10 | AmsGmsAmGmGmAmCfAfAfC | UmsAfsAmUmAmUfUmCmUmGmUf |
| | mAmGmAmAmUmAmUmUmA | UmGmUfCmCfUmCmUmsGmsAm |
| | m | |
| 11 | AmsGmsAmGmGmAmCfAfAfC | UmsAfsAmUmAmUfUmCmUmGmUf |
| | mAmGmAmAmUmAmUmUmA | UmGmUfC(moe)CfUmCmUmsGmsAm |
| | m | |
| 12 | AmsGmsAmGmG(moe)AmCfAfA | UmsAfsAmUmAmUfUmCmUmGmUf |
| | fCmAmGmAmAmUmAmUmUm | UmGmUfC(moe)CfUmCmUmsGmsAm |
| | Am | |
| 13 | CmsUmsGmAmCmAmCfAfAfUf | UmsUfsUmCmUmGfAmGmCmAmUf |
| | GmCmUmCmAmGmAmAmAm | UmGmUfGmUfCmAmGmsAmsUm |
| 14 | CmsUmsGmAmCmAmCfAfAfUf | UmsUfsUmCmUmGfAmGmCmAmUf |
| | GmCmUmCmAmGmAmAmAm | UmGmUfG(moe)UfCmAmGmsAmsUm |
| 15 | CmsUmsGmAmC(moe)AmCfAfA | UmsUfsUmCmUmGfAmGmCmAmUf |
| | fUfGmCmUmCmAmGmAmAmA | UmGmUfG(moe)UfCmAmGmsAmsUm |
| | m | |
| 16 | CmsUmsGmAmCmAmCfAfAfUf | UmsUfsUmCmUmGfAmGmCmAmUf |
| | GmC(moe)UmCmAmGmAmA(m | UmGmUfG(moe)UfCmAmGmsAmsUm |
| | oe)Am | |
| 17 | GmsGmsCmUmUmGmAfUfCfAf | AmsGfsUmAmGmUfUmCmAfUmGm |
| | UmGmAmAmCmUmAmCmUm | AmUmCfA(moe)AfGmCmCmsAmsGm |
| 18 | GmsGmsCmUmUmGmAfUfCfAf | AmsGfsUmAmGmUfUmCmAmUmGm |
| | UmGmAmAmCmUmAmCmUm | AfUmCfA(moe)AfGmCmCmsAmsGm |
| 19 | GmsCmsUmCmCmUmUfAfUfUf | UmsCfsGmUmAmUfAmAmCfAmAm |
| | GmUmUmAmUmAmCmGmAm | UmAmAfG(moe)GfAmGmCmsUmsGm |
| 20 | GmsCmsUmCmCmUmUfAfUfUf | UmsCfsGmUmAmUfAmAmCmAmAm |
| | GmUmUmAmUmAmCmGmAm | UfAmAfG(moe)GfAmGmCmsUmsGm |
| 21 | GmsGmsCmUmUmGmAfUfCfAf | AmsGfsUmAmGmUfUmCmAfUmGm |
| | UmGmAmAmCmUmAmCmUm | AmUmCfAmAfGmCmCmsAmsGm |
| 22 | GmsGmsCmUmUmGmAfUfCfAf | AmsGfsUmAmGmUfUmCmAmUmGm |
| | UmGmAmAmCmUmAmCmUm | AfUmCfAmAfGmCmCmsAmsGm |
| 23 | GmsCmsAmGmCmUmCfCfUfUf | UmsAfsUmAmAmCfAmAmUfAmAm |
| | AmUmUmGmUmUmAmUmAm | GmGmAfGmCfUmGmCmsCmsAm |
| 24 | GmsCmsAmGmCmUmCfCfUfUf | UmsAfsUmAmAmCfAmAmUmAmAm |
| | AmUmUmGmUmUmAmUmAm | GfGmAfGmCfUmGmCmsCmsAm |
| 25 | GmsCmsAmGmCmUmCfCfUfUf | UmsAfsUmAmAmCfAmAmUfAmAm |
| | AmUmUmGmUmUmAmUmAm | GmGmAfG(moe)CfUmGmCmsCmsAm |
| 26 | GmsCmsAmGmCmUmCfCfUfUf | UmsAfsUmAmAmCfAmAmUmAmAm |
| | AmUmUmGmUmUmAmUmAm | GfGmAfG(moe)CfUmGmCmsCmsAm |
| 27 | GmsCmsUmCmCmUmUfAfUfUf | UmsCfsGmUmAmUfAmAmCfAmAm |
| | GmUmUmAmUmAmCmGmAm | UmAmAfGmGfAmGmCmsUmsGm |
| 28 | GmsCmsUmCmCmUmUfAfUfUf | UmsCfsGmUmAmUfAmAmCmAmAm |
| | GmUmUmAmUmAmCmGmAm | UfAmAfGmGfAmGmCmsUmsGm |
| 29 | CmsGmsGmUmAmAmUfGfGfAf | AmsUfsAmAmCmUfCmUmGfUmCmC |
| | CmAmGmAmGmUmUmAmUm | mAmUfUmAfCmCmGmsUmsGm |
| 30 | CmsGmsGmUmAmAmUfGfGfAf | AmsUfsAmAmCmUfCmUmGmUmCm |
| | CmAmGmAmGmUmUmAmUm | CfAmUfUmAfCmCmGmsUmsGm |
| 31 | CmsGmsGmUmAmAmUfGfGfAf | AmsUfsAmAmCmUfCmUmGfUmCmC |
| | CmAmGmAmGmUmUmAmUm | mAmUfU(moe)AfCmCmGmsUmsGm |
| 32 | CmsGmsGmUmAmAmUfGfGfAf | AmsUfsAmAmCmUfCmUmGmUmCm |
| | CmAmGmAmGmUmUmAmUm | CfAmUfU(moe)AfCmCmGmsUmsGm |
| 33 | CmsUmsGmGmCmUmUfGfAfUf | UmsAfsGmUmUmCfUmUmGfAmUm |
| | CmAmAmGmAmAmCmUmAm | CmAmAfGmCfCmAmGmsCmsAm |
| 34 | CmsUmsGmGmCmUmUfGfAfUf | UmsAfsGmUmUmCfUmUmGmAmUm |
| | CmAmAmGmAmAmCmUmAm | CfAmAfGmCfCmAmGmsCmsAm |
| 35 | CmsUmsGmGmCmUmUfGfAfUf | UmsAfsGmUmUmCfUmUmGfAmUm |
| | CmAmAmGmAmAmCmUmAm | CmAmAfG(moe)CfCmAmGmsCmsAm |
| 36 | CmsUmsGmGmCmUmUfGfAfUf | UmsAfsGmUmUmCfUmUmGmAmUm |
| | CmAmAmGmAmAmCmUmAm | CfAmAfG(moe)CfCmAmGmsCmsAm |
| 37 | AmsGmsAmGmGmAmCfAfAfCf | UmsAfsAmUmAmUfUmCmUfGmUm |
| | AmGmAmAmUmAmUmUmAm | UmGmUfCmCfUmCmUmsGmsAm |
| 38 | AmsGmsAmGmGmAmCfAfAfCf | UmsAfsAmUmAmUfUmCmUmGmUm |
| | AmGmAmAmUmAmUmUmAm | UfGmUfCmCfUmCmUmsGmsAm |
| 39 | AmsGmsAmGmGmAmCfAfAfCf | UmsAfsAmUmAmUfUmCmUfGmUm |
| | AmGmAmAmUmAmUmUmAm | UmGmUfC(moe)CfUmCmUmsGmsAm |
| 40 | AmsGmsAmGmGmAmCfAfAfCf | UmsAfsAmUmAmUfUmCmUmGmUm |
| | AmGmAmAmUmAmUmUmAm | UfGmUfC(moe)CfUmCmUmsGmsAm |
| 41 | CmsUmsGmAmCmAmCfAfAfUf | UmsUfsUmCmUmGfAmGmCfAmUm |
| | GmCmUmCmAmGmAmAmAm | UmGmUfGmUfCmAmGmsAmsUm |
| 42 | CmsUmsGmAmCmAmCfAfAfUf | UmsUfsUmCmUmGfAmGmCmAmUm |
| | GmCmUmCmAmGmAmAmAm | UfGmUfGmUfCmAmGmsAmsUm |
| 43 | CmsUmsGmAmCmAmCfAfAfUf | UmsUfsUmCmUmGfAmGmCfAmUm |
| | GmCmUmCmAmGmAmAmAm | UmGmUfG(moe)UfCmAmGmsAmsUm |
| 44 | CmsUmsGmAmCmAmCfAfAfUf | UmsUfsUmCmUmGfAmGmCmAmUm |
| | GmCmUmCmAmGmAmAmAm | UfGmUfG(moe)UfCmAmGmsAmsUm |

### Preparative Example 4: Synthesis of the Sense Strand Conjugated with (CR01008×3) Carrier at 3′ End

### (4.1) Synthesis of Sense Strand

According to the solid-phase nucleic acids synthesis using phosphoramidite method, nucleoside monomers were linked one by one in the direction from 3′ to 5′ according to the nucleotides sequence, starting from the compound CR01008Z connected to the solid phase support in cycles (compound CR01008 was considered as a nucleoside monomer).

The linking of each nucleoside monomer comprised a four-step reaction of deprotection, coupling, capping, and oxidation or sulfurization. The conditions for deprotection, coupling, capping, oxidation or sulfurization reaction, cleavage, deprotection, purification and desalination in the synthesis of the sense strand in this preparative example were the same as those for the synthesis of the sense strand of step (3.1) in preparative example 3.

In this step, a trimeric CR01008 was synthesized during the synthesis of the sense strand, denoted by (CR01008)×3 or (CR01008×3).

The structure formula of the trimeric CRO1008 is given below:

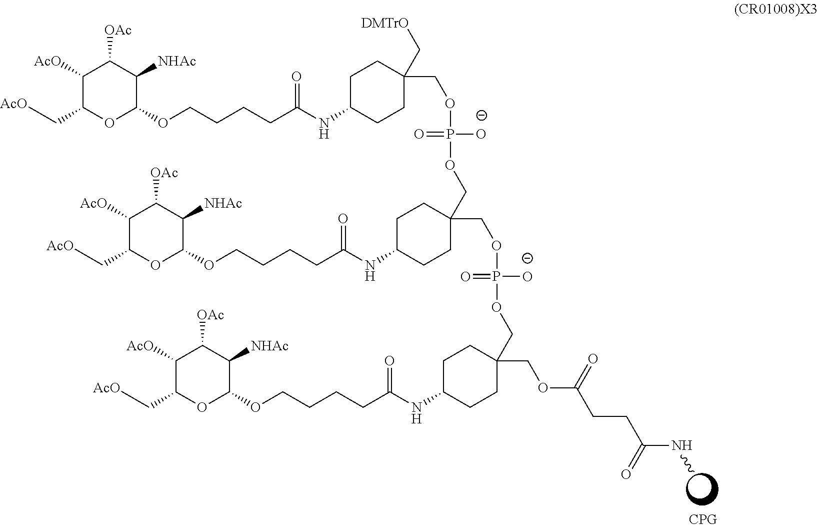

### (5.2) Synthesis of Antisense Strand

The antisense strand of this preparative example was synthesized according to the antisense strand synthesis method shown in step (3.2) of preparative example 3.

(5.3) Synthesis of siRNA Conjugate

The compound conjugated with oligonucleotide of the present preparative example was synthesized according to the method shown in step (3.3) of preparative example 3.

Wherein, when the ligand is trimeric CR01008, the structure formula of the siRNA conjugate is shown below:

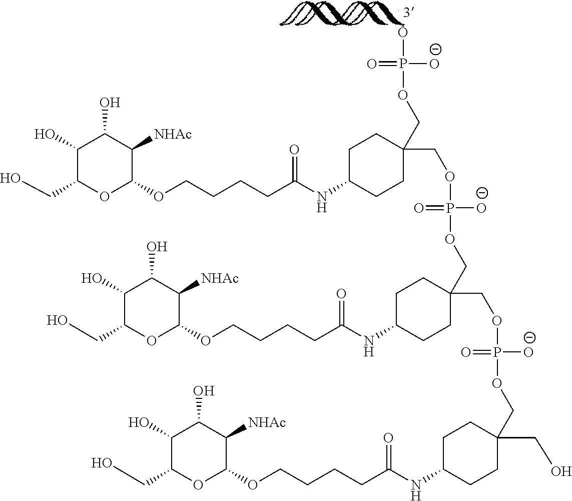

wherein, [symbol] represents siRNA. The (CR01008×3) carrier was conjugated to siRNA at 3′ end of the sense strand. Compounds conjugated with oligonucleotides shown in Table 6 were prepared according to the methods provided in the present disclosure.

TABLE 6
Sequence information of compounds conjugated with
oligonucleotides

| Group | Sense strand (5′-3′) | Antisense strand (5′-3′) |
|---|---|---|
| RZ001031 | GmsGmsCmUmUmGmAfUfCfAf | AmsGfsUmAmGmUfUmCmAmU |
| | UmGmAmAmCmUmAmCmUm | mGfAmUmCfAmAfGmCmCmsA |
| | _(CR01008×3) | msGm |
| RZ001032 | GmsGmsCmUmUmGmAfUfCfAf | AmsGfsUmAmGmUfUmCmAmU |
| | UmGmAmAmCmUmAmCmUm | mGfAmUmCfA(moe)AfGmCmC |
| | _(CR01008×3) | msAmsGm |
| RZ001033 | GmsCmsAmGmCmUmCfCfUfUf | UmsAfsUmAmAmCfAmAmUmA |
| | AmUmUmGmUmUmAmUmAm | mAfGmGmAfGmCfUmGmCmsC |
| | _(CR01008×3) | msAm |
| RZ001034 | GmsCmsAmGmCmUmCfCfUfUf | UmsAfsUmAmAmCfAmAmUmA |
| | AmUmUmGmUmUmAmUmAm | mAfGmGmAfG(moe)CfUmGmC |
| | _(CR01008×3) | msCmsAm |
| RZ001035 | GmsCmsUmCmCmUmUfAfUfUf | UmsCfsGmUmAmUfAmAmCmA |
| | GmUmUmAmUmAmCmGmAm | mAfUmAmAfGmGfAmGmCmsU |
| | _(CR01008×3) | msGm |
| RZ001036 | CmsGmsGmUmAmAmUfGfGfAf | AmsUfsAmAmCmUfCmUmGmU |
| | CmAmGmAmGmUmUmAmUm | mCfCmAmUfUmAfCmCmGmsU |
| | _(CR01008×3) | msGm |
| RZ001037 | CmsGmsGmUmAmAmUfGfGfAf | AmsUfsAmAmCmUfCmUmGmU |
| | CmAmGmAmGmUmUmAmUm | mCfCmAmUfT(moe)AfCmCm |
| | _(CR01008×3) | GmsUmsGm |
| RZ001038 | CmsUmsGmGmCmUmUfGfAfUf | UmsAfsGmUmUmCfUmUmGmA |
| | CmAmAmGmAmAmCmUmAm | mUfCmAmAfGmCfCmAmGmsC |
| | _(CR01008×3) | msAm |
| RZ001039 | CmsUmsGmGmCmUmUfGfAfUf | UmsAfsGmUmUmCfUmUmGmA |
| | CmAmAmGmAmAmCmUmAm | mUfCmAmAfG(moe)CfCmAmG |
| | _(CR01008×3) | msCmsAm |
| RZ001040 | AmsGmsAmGmGmAmCfAfAfC | UmsAfsAmUmAmUfUmCmUmG |
| | mAmGmAmAmUmAmUmUmA | mUfUmGmUfCmCfUmCmUmsG |
| | m_(CR01008×3) | msAm |
| RZ001041 | AmsGmsAmGmGmAmCfAfAfC | UmsAfsAmUmAmUfUmCmUmG |
| | mAmGmAmAmUmAmUmUmA | mUfUmGmUfC(moe)CfUmCmU |
| | m_(CR01008×3) | msGmsAm |
| RZ001042 | AmsGmsAmGmG_(moe)AmCfAf | UmsAfsAmUmAmUfUmCmUmG |
| | AfCmAmGmAmAmUmAmUmU | mUfUmGmUfC(moe)CfUmCmU |
| | mAm_(CR01008×3) | msGmsAm |
| RZ001043 | CmsUmsGmAmCmAmCfAfAfUf | UmsUfsUmCmUmGfAmGmCmA |
| | GmCmUmCmAmGmAmAmAm | mUfUmGmUfGmUfCmAmGmsA |
| | _(CR01008×3) | msUm |
| RZ001044 | CmsUmsGmAmCmAmCfAfAfUf | UmsUfsUmCmUmGfAmGmCmA |
| | GmCmUmCmAmGmAmAmAm | mUfUmGmUfG(moe)UfCmAmG |
| | _(CR01008×3) | msAmsUm |
| RZ001045 | CmsUmsGmAmC_(moe)AmCfAfA | UmsUfsUmCmUmGfAmGmCmA |
| | fUfGmCmUmCmAmGmAmAmA | mUfUmGmUfG(moe)UfCmAmG |
| | m_(CR01008×3) | msAmsUm |
| RZ001046 | CmsUmsGmAmCmAmCfAfAfUf | UmsUfsUmCmUmGfAmGmCmA |
| | GmC_(moe)UmCmAmGmAmA_ | mUfUmGmUfG(moe)UfCmAmG |
| | (moe)Am_(CR01008×3) | msAmsUm |
| RZ001047 | GmsGmsCmUmUmGmAfUfCfAf | AmsGfsUmAmGmUfUmCmAfU |
| | UmGmAmAmCmUmAmCmUm | mGmAmUmCfA(moe)AfGmCmC |
| | _(CR01008×3) | msAmsGm |
| RZ001048 | GmsGmsCmUmUmGmAfUfCfAf | AmsGfsUmAmGmUfUmCmAmU |
| | UmGmAmAmCmUmAmCmUm | mGmAfUmCfA(moe)AfGmCmC |
| | _(CR01008×3) | msAmsGm |
| RZ001049 | GmsCmsUmCmCmUmUfAfUfUf | UmsCfsGmUmAmUfAmAmCfA |
| | GmUmUmAmUmAmCmGmAm | mAmUmAmAfG(moe)GfAmGmC |
| | _(CR01008×3) | msUmsGm |
| RZ001050 | GmsCmsUmCmCmUmUfAfUfUf | UmsCfsGmUmAmUfAmAmCmA |
| | GmUmUmAmUmAmCmGmAm | mAmUfAmAfG(moe)GfAmGmC |
| | _(CR01008×3) | msUmsGm |
| RZ001051 | GmsGmsCmUmUmGmAfUfCfAf | AmsGfsUmAmGmUfUmCmAfU |
| | UmGmAmAmCmUmAmCmUm | mGmAmUmCfAmAfGmCmCms |
| | _(CR01008×3) | AmsGm |
| RZ001052 | GmsGmsCmUmUmGmAfUfCfAf | AmsGfsUmAmGmUfUmCmAmU |
| | UmGmAmAmCmUmAmCmUm | mGmAfUmCfAmAfGmCmCmsA |
| | _(CR01008×3) | msGm |
| RZ001053 | GmsCmsAmGmCmUmCfCfUfUf | UmsAfsUmAmAmCfAmAmUfA |
| | AmUmUmGmUmUmAmUmAm | mAmGmGmAfGmCfUmGmCms |
| | _(CR01008×3) | CmsAm |
| RZ001054 | GmsCmsAmGmCmUmCfCfUfUf | UmsAfsUmAmAmCfAmAmUmA |
| | AmUmUmGmUmUmAmUmAm | mAmGfGmAfGmCfUmGmCmsC |
| | _(CR01008×3) | msAm |
| RZ001055 | GmsCmsAmGmCmUmCfCfUfUf | UmsAfsUmAmAmCfAmAmUfA |
| | AmUmUmGmUmUmAmUmAm | mAmGmGmAfG(moe)CfUmGmC |
| | _(CR01008×3) | msCmsAm |
| RZ001056 | GmsCmsAmGmCmUmCfCfUfUf | UmsAfsUmAmAmCfAmAmUmA |
| | AmUmUmGmUmUmAmUmAm | mAmGfGmAfG(moe)CfUmGmC |
| | _(CR01008×3) | msCmsAm |
| RZ001057 | GmsCmsUmCmCmUmUfAfUfUf | UmsCfsGmUmAmUfAmAmCfA |
| | GmUmUmAmUmAmCmGmAm | mAmUmAmAfGmGfAmGmCms |
| | _(CR01008×3) | UmsGm |
| RZ001058 | GmsCmsUmCmCmUmUfAfUfUf | UmsCfsGmUmAmUfAmAmCmA |
| | GmUmUmAmUmAmCmGmAm | mAmUfAmAfGmGfAmGmCmsU |
| | _(CR01008×3) | msGm |
| RZ001059 | CmsGmsGmUmAmAmUfGfGfAf | AmsUfsAmAmCmUfCmUmGfU |
| | CmAmGmAmGmUmUmAmUm | mCmCmAmUfUmAfCmCmGms |
| | _(CR01008×3) | UmsGm |
| RZ001060 | CmsGmsGmUmAmAmUfGfGfAf | AmsUfsAmAmCmUfCmUmGmU |
| | CmAmGmAmGmUmUmAmUm | mCmCfAmUfUmAfCmCmGmsU |
| | _(CR01008×3) | msGm |
| RZ001061 | CmsGmsGmUmAmAmUfGfGfAf | AmsUfsAmAmCmUfCmUmGfU |
| | CmAmGmAmGmUmUmAmUm | mCmCmAmUfU(moe)AfCmCmG |
| | _(CR01008×3) | msUmsGm |
| RZ001062 | CmsGmsGmUmAmAmUfGfGfAf | AmsUfsAmAmCmUfCmUmGmU |
| | CmAmGmAmGmUmUmAmUm | mCmCfAmUfU(moe)AfCmCmG |
| | _(CR01008×3) | msUmsGm |
| RZ001063 | CmsUmsGmGmCmUmUfGfAfUf | UmsAfsGmUmUmCfUmUmGfA |
| | CmAmAmGmAmAmCmUmAm | mUmCmAmAfGmCfCmAmGms |
| | _(CR01008×3) | CmsAm |
| RZ001064 | CmsUmsGmGmCmUmUfGfAfUf | UmsAfsGmUmUmCfUmUmGmA |
| | CmAmAmGmAmAmCmUmAm | mUmCfAmAfGmCfCmAmGmsC |
| | _(CR01008×3) | msAm |
| RZ001065 | CmsUmsGmGmCmUmUfGfAfUf | UmsAfsGmUmUmCfUmUmGfA |
| | CmAmAmGmAmAmCmUmAm | mUmCmAmAfG(moe)CfCmAmG |
| | _(CR01008×3) | msCmsAm |
| RZ001066 | CmsUmsGmGmCmUmUfGfAfUf | UmsAfsGmUmUmCfUmUmGmA |
| | CmAmAmGmAmAmCmUmAm | mUmCfAmAfG(moe)CfCmAmG |
| | _(CR01008×3) | msCmsAm |
| RZ001067 | AmsGmsAmGmGmAmCfAfAfCf | UmsAfsAmUmAmUfUmCmUfG |
| | AmGmAmAmUmAmUmUmAm | mUmUmGmUfCmCfUmCmUms |
| | _(CR01008×3) | GmsAm |
| RZ001068 | AmsGmsAmGmGmAmCfAfAfCf | UmsAfsAmUmAmUfUmCmUmG |
| | AmGmAmAmUmAmUmUmAm | mUmUfGmUfCmCfUmCmUmsG |
| | _(CR01008×3) | msAm |
| RZ001069 | AmsGmsAmGmGmAmCfAfAfCf | UmsAfsAmUmAmUfUmCmUfG |
| | AmGmAmAmUmAmUmUmAm | mUmUmGmUfC(moe)CfUmCmU |
| | _(CR01008×3) | msGmsAm |
| RZ001070 | AmsGmsAmGmGmAmCfAfAfCf | UmsAfsAmUmAmUfUmCmUmG |
| | AmGmAmAmUmAmUmUmAm | mUmUfGmUfC(moe)CfUmCmU |
| | _(CR01008×3) | msGmsAm |
| RZ001071 | CmsUmsGmAmCmAmCfAfAfUf | UmsUfsUmCmUmGfAmGmCfA |
| | GmCmUmCmAmGmAmAmAm | mUmUmGmUfGmUfCmAmGms |
| | _(CR01008×3) | AmsUm |
| RZ001072 | CmsUmsGmAmCmAmCfAfAfUf | UmsUfsUmCmUmGfAmGmCmA |
| | GmCmUmCmAmGmAmAmAm | mUmUfGmUfGmUfCmAmGmsA |
| | _(CR01008×3) | msUm |
| RZ001073 | CmsUmsGmAmCmAmCfAfAfUf | UmsUfsUmCmUmGfAmGmCfA |
| | GmCmUmCmAmGmAmAmAm | mUmUmGmUfG(moe)UfCmAmG |
| | _(CR01008×3) | msAmsUm |
| RZ001074 | CmsUmsGmAmCmAmCfAfAfUf | UmsUfsUmCmUmGfAmGmCmA |
| | GmCmUmCmAmGmAmAmAm | mUmUfGmUfG(moe)UfCmAmG |
| | _(CR01008×3) | msAmsUm |

“_(CR01008×3)” indicates that the ligand represented by (CR01008×3) is conjugated to the 3′ end of the sense strand.

Unless otherwise specified, the meanings of the base compositions and modifications described in various examples of the present disclosure are as follows: uppercase letters A, U, G, C and T represent the base composition of the nucleotides, lowercase letter m represents that the nucleotide adjacent to the left side of the letter m is a 2′-O-methyl-modified (also known as 2′-methoxy-modified) nucleotide, lowercase letter f represents that the nucleotide adjacent to the left side of the letter f is a 2′-fluoro-modified nucleotide, lowercase letter d represents that the nucleotide adjacent to the left side of the letter d is a 2′-deoxy-modified ribonucleic acid (also known as: deoxyribonucleic acid), (moe) represents that the nucleotide adjacent to the left side of the combination sign (moe) is a 2′-O-methoxyethyl-modified nucleotide, and lowercase letter s represents that the two nucleotides adjacent to both sides of the letter s are linked by a phosphorothioate linkage.

### Biological Assay

In the present disclosure, unless otherwise indicated, the siRNA sequences used in the present disclosure were synthesized by Kunshan Alltest Biotech Co., Ltd.; the PCR primers used in the present disclosure were synthesized by Sangon Biotech (Shanghai) Co., Ltd.; the HEK293-LPA stable cell line used in the present disclosure was a gift from Beijing University of Technology; the experimental animals including BALB/c mice used in the present disclosure were purchased from Zhejiang Vital River Laboratory Animal Technology Co., Ltd.; the experimental animals including ICR mice used in the present disclosure were purchased from Zhejiang Vital River Laboratory Animal Technology Co., Ltd.; the hApo(a) transgenic mice used in the present disclosure were constructed by Saiye (Suzhou) Biotechnology Co., Ltd. entrusted by Suzhou xuanjing Biotechnology Co., Ltd.; the serum samples of transgenic mice in the present disclosure were detected by human lipoprotein a ELISA Kit.

In the present disclosure, unless otherwise indicated, for data from real-time PCR assays related to in vivo activity assays, the AACt method was used to relatively quantify the mRNA expression of the target gene in each test group, which was calculated as follows:

$$Δ Ct ( test group ) = Ct ( target gene in test group ) - ( Ct ( internal reference gene in test group ) ;$$

$$Δ Ct ( control group ) = Ct ( target gene in control group ) - Ct ( internal reference gene in control group ) ;$$

$$ΔΔ Ct ( test group ) = Δ Ct ( test group ) - Δ Ct ( mean of control group ) ;$$

$$ΔΔ Ct ( control group ) = Δ Ct ( control group ) - Δ Ct ( mean of control group ) ;$$

The mRNA expression level of the target gene in the test group was normalized using the control group as the baseline, and the mRNA expression level of the target gene in the control group was defined as 100%;

$$Relative retained expression level of mRNA of target gene in test group = {2}^{- ΔΔ Ct ( test group )} \times 100 \% ;$$

$$Inhibition rate of mRNA of target gene in test group = 100 \% - relative retained expression level of mRNA of target gene in test group .$$

Data for the in vivo activity assay of Apo(a) protein at different time points covered by the present disclosure are processed as shown below.

$$Relatively remained expression level of protein = \frac{{D}_{x}}{D 0} \times 100 \%$$

In the present disclosure, unless otherwise indicated, all data for in vivo activity assay is presented as X̅±SD (X̅±STDEV (standard deviation)). The experimental data were graphed and analyzed using GraphPad Prism 8.0 software.

### Example 1 In Vivo Activity Evaluation of Compounds with (CR01008)×3 Conjugated at 3′ End of the Sense Strand in HDI Mice

In this example, the inhibitory activity of siRNA conjugate (RZ001031-RZ001046) on target gene LPA in HDI mice was evaluated by using the hydrodynamic injection model of balb/c mice, with RZ000001 as the negative control and Olpasiran as the positive control. RZ000001 and Olpasiran are shown in the following table:

| | Sense strand | Antisense strand |
|---|---|---|
| Group | (5′-3′) | (5′-3′) |
| RZ000001 | UmsUmsCmUmCmCmGfA | AmsCfsGmUmGmAfCm |
| | fAfCmGmUmGmUmCmAm | AmCmGmUmUmCmGfGm |
| | CmGmUmL96 | AfGmAmAmsCmsUm |
| Olpasiran | (NAG25)sCmsAmGmCm | UmsCfsGmUfAmUfAm |
| | CmCmCmUmUfAfUfUmGm | AmCmAmAmUfAmAfGm |
| | UmUmAmUmAmCm | GfGmGfCmsUfsGm |
| | Gms(invdA) | |

### Animal Grouping, Administration and Collection of Tissue Sample

BALB/c mice were randomly grouped according to body weight (all female), with 5 mice in each group. The same pcDNA-CMV-RG001 plasmid and mouse hydrodynamic injection model construction method were used to establish the mouse HDI model. Each test group was given a predetermined dose of drug, and a PBS control group was set. The administration dose was calculated based on body weight for all mice, and the drug was given by a single subcutaneous injection in the abdomen, wherein drug was given as solutions of 0.1 mg siRNA/mL PBS, the dosing volume was 10 mL/kg body weight of mouse, that is, the dosing amount was 1 mg siRNA/kg body weight of mouse. The PBS control group was given the same volume of PBS solution without containing drug. The day of administration was denoted as day 0 (denoted as DO), plasmid injection was performed on day 3 (denoted as D3) after administration, and all mice were executed on day 4 (denoted as D4). The executed mice were separately subjected to gross necropsy. The liver tissues of each mouse were collected, which were cut into about 2 mm³ small pieces, and stored with RNA later.

For each mouse, the liver tissue sample was taken out of the RNA later, and homogenized in an automatic tissue homogenizer-Tissuelyser II for 60 seconds. According to the standard protocol for total RNA extraction, the total RNA was extracted using an automatic nucleic acid extractor (purchased from Zhejiang Hanwei Technology Co., Ltd.) and a nucleic acid extraction kit (purchased from Zhejiang Hanwei Technology Co., Ltd., GO-MNTR-100).

For each mouse, 1 g of the total RNA was taken, 20 μL of reverse transcription system was prepared using a reverse transcription kit (Thermo Fisher Scientific, RevertAid First Strand cDNA Synthesis Kit, K1622) and Oligo (dT)18 primer for reverse transcription according the instructions of the reverse transcription kit, and a reverse transcription reaction was carried out. After the reaction was completed, 60 μL of RNase-Free water was added to the reverse transcription system to obtain a cDNA solution. Next, the mRNA expression of the target gene in animals was measured in a fluorescence quantitative PCR instrument (Bio-Rad, CFX Opus 384) using a real-time fluorescence quantitative PCR kit (Thermo Fisher Scientific, TaqMan Fast Advanced Master Mix, 4444557). In the real-time fluorescence quantitative PCR assay, the Nero gene on plasmid skeleton was used as the internal reference gene, and primers for the target gene and the internal reference gene were used to detect the target gene and the internal reference gene respectively. Sequences of primers used are shown in Table 7.

TABLE 7
Primer sequence

| | | | | Fluorescent |
|---|---|---|---|---|
| Gene | | Primer | Primer sequence | group |
| Target | LPA | Upstream | 5′-GACACGATGCTCAGATGC-3′ | / |
| gene | | primer | (SEQ ID No. 15) | |
| | | Downstream | 5′-GGGGTTTCCTCAGTCAGT-3′ | / |
| | | primer | (SEQ ID No. 16) | |
| | | Probe | 5′-CGTCCCTCCGAATGTTA-3′ | 5′FAM; |
| | | primer | (SEQ ID No. 17) | 3′MGB |
| Internal | Nero | Upstream | 5′-CGTTGGCTACCCGTGATATT-3′ | / |
| reference | | primer | (SEQ ID No. 18) | |
| gene | | Downstream | 5′-CTCGTCAAGAAGGCGATAGA | / |
| | | primer | AG-3′ | |
| | | | (SEQ ID No. 19) | |
| | | Probe | 5′- | 5′VIC; |
| | | primer | CCGCTTCCTCGTGCTTTACGGTAT- | 3′MGB |
| | | | 3′ (SEQ ID No. 20) | |

A 10 μL real-time PCR reaction system per PCR reaction well was prepared according to the protocol documented in the instructions of the real-time fluorescence quantitative PCR kit, and each reaction system consisted of 4 μL of cDNA solution obtained by the reverse transcription reaction described above, 5 L of TaqMan™ Fast Advanced Master Mix (2×), 0.15 μL of 10 M upstream primer, 0.15 μL of 10 M downstream primer, 0.15 μL of 10 M probe primer and 0.55 μL of RNase-Free H₂O. The prepared reaction system was placed in a real-time fluorescence quantitative PCR instrument (Bio-Rad, CFX Opus 384), and amplified by two-step method. The amplification procedure was 2 min at 50° C., predenaturation at 95° C. for 20 seconds, denaturation at 95° C. for 3 seconds, annealing at 60° C. and extension for 30 seconds. The above denaturation, annealing and extension processes were repeated for 40 cycles. In this real-time fluorescence quantitative PCR assay, the mRNA expression level of the target gene in each test group and the inhibition rate was relatively quantified using the ΔΔCt method as described in the examples.

TABLE 8
Inhibitory activity of the compounds described in this example
on target gene in BALB/c-HDI mice after administration

1 mg/kg

| | % Relatively remained | |
|---|---|---|
| group | expression level | STDEV |
| PBS | 100.00 | 16.50 |
| RZ000001 | 118.45 | 30.66 |
| Olpasiran | 26.74 | 12.05 |
| RZ001031 | 18.76 | 3.48 |
| RZ001032 | 19.61 | 5.91 |
| RZ001033 | 19.71 | 4.97 |
| RZ001034 | 18.39 | 1.92 |
| RZ001035 | 25.13 | 9.81 |
| RZ001036 | 56.45 | 19.15 |
| RZ001037 | 52.72 | 14.26 |
| RZ001038 | 30.55 | 9.83 |
| RZ001039 | 30.85 | 5.25 |
| RZ001040 | 42.92 | 5.05 |
| RZ001041 | 41.57 | 15.93 |
| RZ001042 | 55.65 | 17.53 |
| RZ001043 | 35.69 | 8.05 |
| RZ001044 | 42.63 | 35.01 |
| RZ001045 | 43.41 | 13.23 |
| RZ001046 | 36.96 | 12.38 |

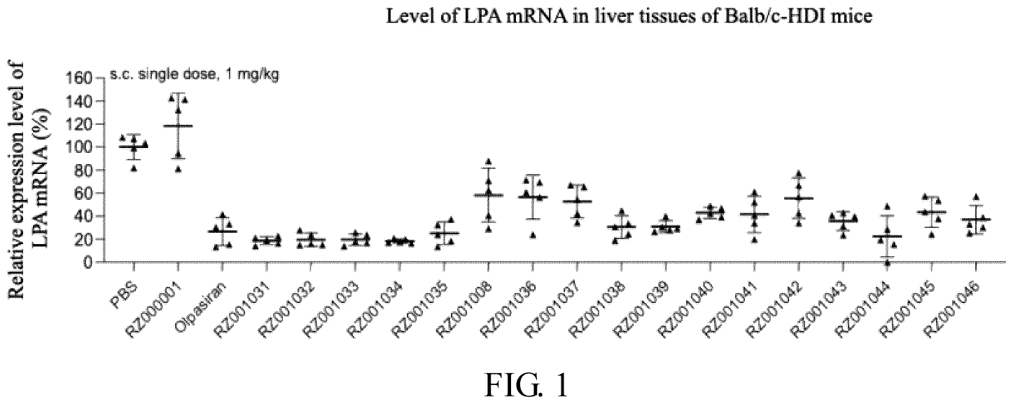

The results of example 1 showed that siRNA conjugates with different modified CR01008 carrier significantly inhibited the LPA mRNA expression in liver tissues at the dose of 1 mg/kg, and the inhibitory effect of RZ001031, RZ001032, RZ001033, RZ001034 on mRNA were better than Olpasiran (FIG. 1 and Table 8).

### Example 2 In Vivo Pharmacodynamic Evaluation of Compounds with (CR01008)×3 Conjugated at 3′ End of the Sense Strand in hApo(a) Transgenic Mice

This example measured the Apo(a) protein expression in serums of mice at different time points after a single administration of CR01008 carrier conjugate RZ001031, RZ001032, RZ001033, RZ001034, RZ001035, RZ001038, RZ001039 and RZ0030044, by enzyme-linked immunosorbent assay (ELISA), with Olpasiran as the control.

### Animal Grouping, Administration and Collection of Tissue Sample

The hApo(a) transgenic mice (constructed by Saiye (Suzhou) Biotechnology Co., Ltd. entrusted by Suzhou xuanjing Biotechnology Co., Ltd.) were grouped according to the hApo(a) protein level in the serum of mice. Each test group was given a predetermined dose of drug, and a PBS control group was set. The administration dose was calculated based on body weight for all mice, and the drug was given by a single subcutaneous injection in the abdomen, wherein drug was given as solutions of 0.1 mg siRNA/mL PBS, the dosing volume was 10 mL/kg body weight of mouse, that is, the dosing amount was 1 mg siRNA/kg body weight of mouse. The PBS control group was given the same volume of PBS solution without containing drug. The serums were collected from the remaining mice of all groups before administration (denoted by pre), on day 7 after administration (denoted by D7), on day 14 after administration (denoted by D14), on day 21 after administration (denoted by D21), on day 28 after administration (denoted by D28), on day 35 after administration (denoted by D35), on day 42 after administration (denoted by D42), on day 49 after administration (denoted by D49), on day 56 after administration (denoted by D56), on day 63 after administration (denoted by D63), on day 70 after administration (denoted by D70), and the Apo(a) protein expression level was measured with the human Lipoprotein AELISA kit (Abcam, ab212165).

### Changes of Human Apo (a) Protein in Transgenic Mice

TABLE 9
Changes of hApo(a) protein in transgenic mice, after administration of the compounds described in this example

| group | | Olpasiran | RZ001031 | RZ001032 | RZ001033 | RZ001034 | RZ001035 | RZ001038 | RZ001039 | RZ001044 |
|---|---|---|---|---|---|---|---|---|---|---|
| Pre | % Relatively remained | 100 | 100 | 100 | 100 | 100 | 100 | 100 | 100 | 100 |
| | expression level | | | | | | | | | |
| | STDEV | 0 | 0 | 0 | 0 | 0 | 0 | 0 | 0 | 0 |
| D 7 | % Relatively remained | 31.34 | 13.22 | 12.13 | 17.14 | 16.02 | 19.78 | 34.65 | 24.19 | 63.46 |
| | expression level | | | | | | | | | |
| | STDEV | 8.42 | 0.76 | 10.64 | 5.93 | 3.49 | 3.43 | 19.34 | 7.81 | 11.96 |
| D 14 | % Relatively remained | 25.47 | 12.98 | 10.11 | 19.73 | 15.28 | 15.94 | 31.28 | 27.56 | 63.42 |
| | expression level | | | | | | | | | |
| | STDEV | 15.68 | 4.01 | 10.34 | 4.78 | 3.5 | 0.32 | 17.01 | 10.25 | 15.78 |
| D 21 | % Relatively remained | 27.3 | 17.29 | 11.57 | 27.37 | 35.24 | 12.74 | 35.13 | 76.49 | 86.04 |
| | expression level | | | | | | | | | |
| | STDEV | 10.79 | 3.36 | 8.02 | 3.81 | 6.3 | 3.89 | 12.29 | 65.77 | 35.77 |
| D 28 | % Relatively remained | 29.7 | 22.44 | 18.7 | 54.23 | 36.14 | 25.79 | 48.93 | 72.82 | 89.61 |
| | expression level | | | | | | | | | |
| | STDEV | 13.61 | 3.41 | 12.82 | 20.77 | 5.41 | 12.89 | 14.9 | 55.57 | 33.11 |
| D 35 | % Relatively remained | 45.84 | 44.9 | 25.73 | 86.26 | 60.75 | 35.99 | 51.65 | 77.01 | 99.11 |
| | expression level | | | | | | | | | |
| | STDEV | 20.28 | 16.52 | 19.46 | 20.13 | 10.34 | 6.68 | 19.42 | 13.18 | 46.05 |
| D 42 | % Relatively remained | 57.32 | 61.98 | 34.36 | 67.63 | 68.89 | 57.19 | 51.9 | 68.08 | 95.85 |
| | expression level | | | | | | | | | |
| | STDEV | 23.61 | 6.6 | 20.33 | 15.35 | 14.37 | 26.97 | 11.72 | 14.51 | 37.07 |
| D 49 | % Relatively remained | 73.8 | 75.24 | 44.1 | 72.38 | 90.2 | 71.34 | 74.03 | 79.8 | 97.64 |
| | expression level | | | | | | | | | |
| | STDEV | 5.7 | 26.67 | 17.83 | 20.67 | 39 | 15.18 | 26.23 | 24.12 | 13.91 |
| D 56 | % Relatively remained | 70.16 | 96.05 | 51.24 | 84.3 | 103.9 | 80.16 | 77.67 | 95.69 | 105.44 |
| | expression level | | | | | | | | | |
| | STDEV | 15.51 | 52.19 | 15.28 | 5.17 | 36.74 | 11.89 | 34.09 | 24.59 | 33.07 |
| D 63 | % Relatively remained | 83.65 | 87.85 | 66.91 | — | — | 87.14 | — | — | — |
| | expression level | | | | | | | | | |
| | STDEV | 14.36 | 12.54 | 17.21 | — | — | 5.84 | — | — | — |
| D 70 | % Relatively remained | 89.32 | 87.07 | 73.73 | — | — | 98.22 | — | — | — |
| | expression level | | | | | | | | | |
| | STDEV | 15.19 | 18.58 | 18.75 | — | — | 11.49 | — | — | — |

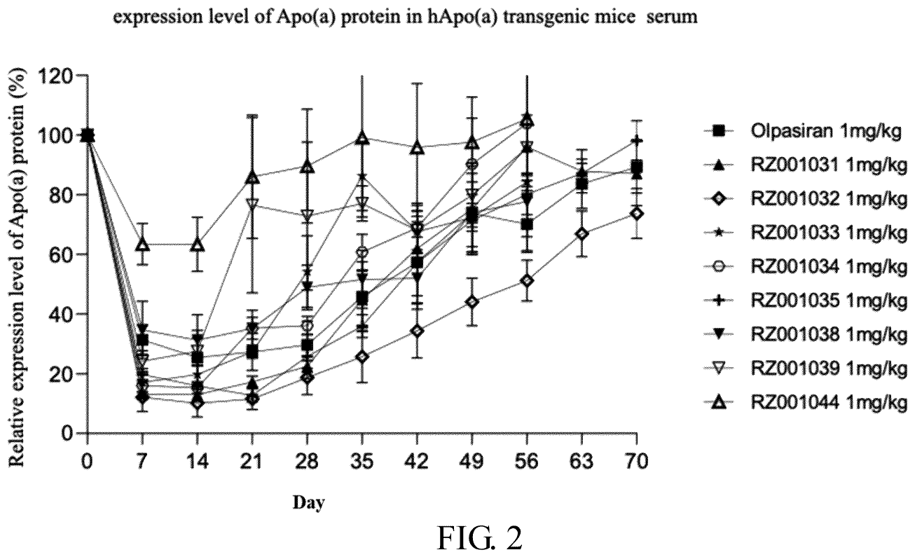

The results of example 2 showed that RZ001031, RZ001032, RZ001033, RZ001034, RZ001035, RZ001038 and RZ001039 significantly reduced the level ofApo(a) protein in transgenic mice at a single dose of 1 mg/kg; on day 28, RZ010032 reduced Apo(a) protein levels by 90%, while Olpasiran reduced Apo(a) protein levels by 75%; on day 56, RZ010032 reduced Apo(a) protein levels by 50%, while Olpasiran reduced Apo(a) protein levels by 30%; it was observed that on day 70, Apo(a) protein levels in the RZ010032 group still had not returned to the pre-dose level (FIG. 2 and Table 9).

The above specific embodiments are merely schematic illustrations of the contents of the present disclosure and do not represent limitations of the contents of the present disclosure. For those of ordinary skills in the art, without departing from the spirit and substance of the present disclosure, various variations and modifications may be made, which are also regarded as the scope of protection of the present disclosure.

1. A compound having a structure represented by formula (I), or a pharmaceutically acceptable salt thereof: 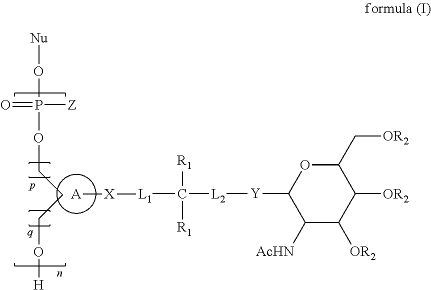
wherein, in the structure,
each A is independently an unsubstituted or substituted 4-to 10-membered aliphatic ring,
n is selected from the group consisting of 1, 2, 3 and 4,
each Z is independently selected from the group consisting of hydroxyl and mercapto,
each p is independently selected from the group consisting of 1, 2 and 3,
each q is independently selected from the group consisting of 1, 2 and 3,
each X is independently selected from the group consisting of NH, O and S,
each L₁ is independently selected from the group consisting of 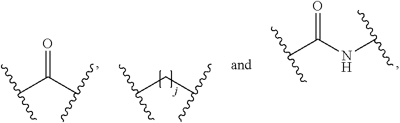
wherein j is selected from the group consisting of 1, 2, 3, 4, 5, 6, 7, 8, 9 and 10,
each R₁ is independently selected from the group consisting of H, C₁-C₆ alkyl, C₁-C₆ haloalkyl and C₁-C₆ alkoxy,
each L₂ is independently selected from the group consisting of C₁-C₃₀ alkylidene and 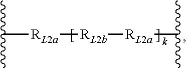
wherein each R_{L2a} is independently C₁-C₁₀ alkylidene, each R_{L2b} is independently selected from the group consisting of O, S, NH and —NH—C(O)—, and k is selected from the group consisting of 1, 2, 3, 4, 5, 6, 7, 8, 9 and 10,
each Y is independently selected from the group consisting of NH, O and S, and
each R₂ is independently selected from the group consisting of: H, 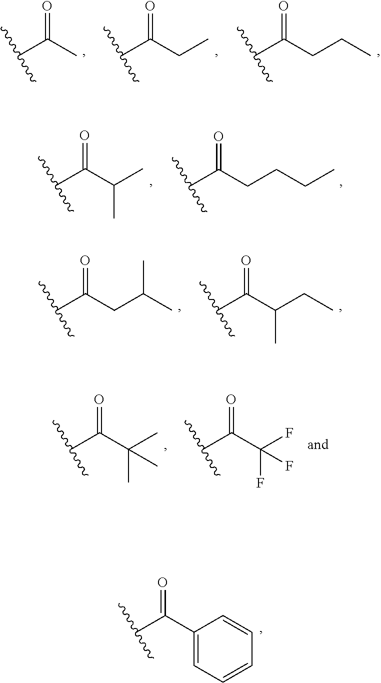
wherein Nu represents a double-stranded oligonucleotide or a pharmaceutically acceptable salt thereof for reducing the expression of intracellular LPA gene, the double-stranded oligonucleotide comprises a sense strand and an antisense strand, wherein the sense strand and the antisense strand form a double-stranded region, the antisense strand comprises a complementary region that has complementarity to a target sequence of LPA mRNA, and the target sequence is selected from a nucleotide region comprising 14 to 35 consecutive nucleotides on the LPA mRNA.

2. The compound according to claim 1, wherein the compound has a structure represented by formula (II), or the pharmaceutically acceptable salt thereof, 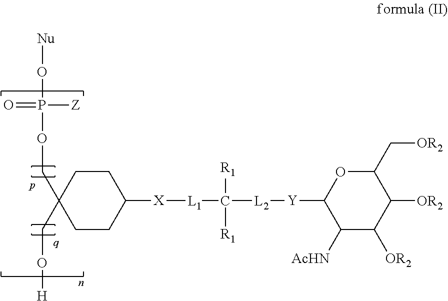
in formula (II), p, q, n, Z, X, Y, L₁, L₂ and R₁ are as defined in claim 1, and R₂ is H.

3. The compound according to claim 1, wherein the compound has a structure represented by formula (III), or the pharmaceutically acceptable salt thereof, 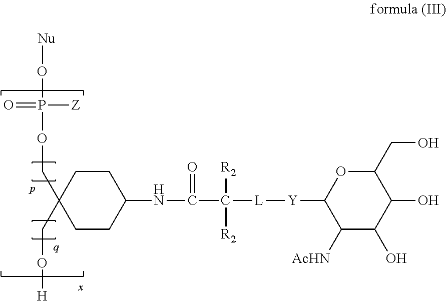
in formula (III), m is selected from the group consisting of 1, 2, 3 and 4, and the other substituents are as defined in claim 1;
optionally, the compound has a structure represented by formula (IV), or the pharmaceutically acceptable salt thereof, 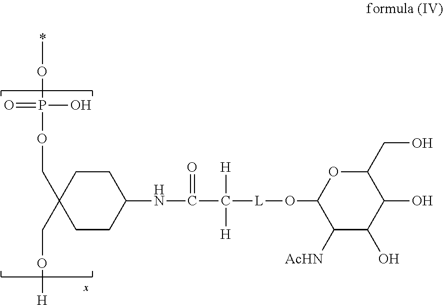
in formula (IV), Nu is as defined in claim 1, m is selected from the group consisting of 1, 2, 3 and 4, and L₂ is dependently selected from the group consisting of

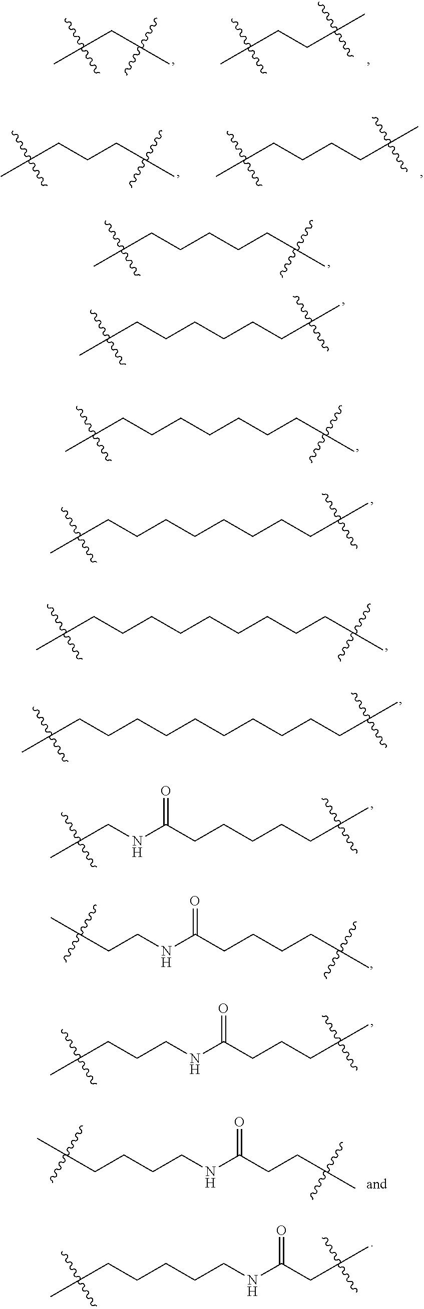

4. The compound according to claim 1, wherein the compound has a structure selected from the group consisting of 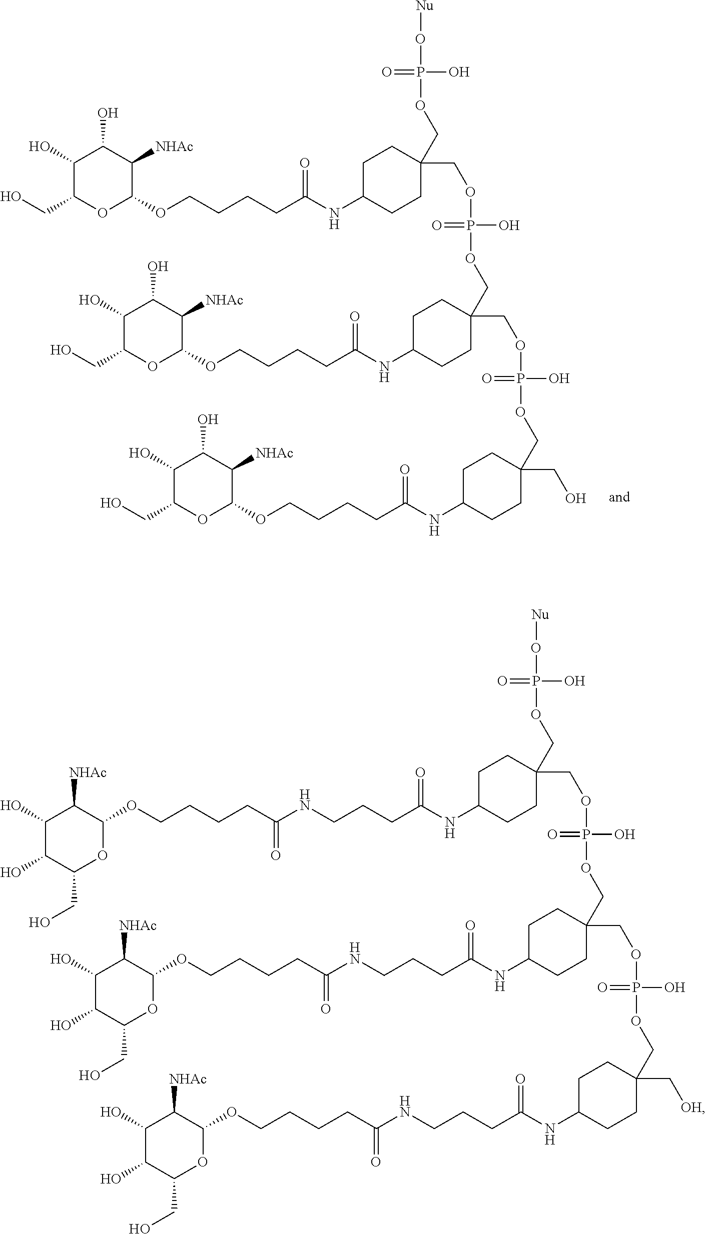
or the pharmaceutically acceptable salt thereof,
wherein Nu is as defined in claim 1, and in the above oligonucleotide-conjugated compound, the sense strand of Nu is connected at 3′ end to a phosphate group.

5. The compound according to claim 1, wherein the sense strand of the double-stranded oligonucleotide represented by Nu comprises a nucleotide sequence of or differing by 1, 2 or 3 nucleotides from at least 17, at least 18, or at least 19 consecutive nucleotides in any one of the sequences set forth in SEQ ID NOs: 1, 3, 5, 7, 9, 11 and 13; and/or, the antisense strand comprises a nucleotide sequence of or differing by 1, 2 or 3 nucleotides from at least 17, at least 18, or at least 19 consecutive nucleotides in any one of the sequences set forth in SEQ ID NOs: 2, 4, 6, 8, 10, 12 and 14; optionally, according to the 5′-3′direction, the antisense strand of the double-stranded oligonucleotide represented by Nu comprises a nucleotide sequence of or differing by 1, 2 or 3 nucleotides from 1st to 19th consecutive nucleotides in any one of the sequences set forth in SEQ ID NOs: 2, 4, 6, 8, 10, 12 and 14;
optionally, the sense strand of the double-stranded oligonucleotide comprises a nucleotide sequence of or differing by 1 or 2 nucleotides from any one of the sequences set forth in SEQ ID NOs: 1, 3, 5, 7, 9, 11 and 13, and/or, the antisense strand of the double-stranded oligonucleotide represented by Nu comprises a nucleotide sequence of or differing by 1 or 2 nucleotides from any one of the sequences set forth in SEQ ID NOs: 2, 4, 6, 8, 10, 12 and 14;
optionally, the double-stranded oligonucleotide is one or more selected from the group consisting of:
1) an antisense strand having a nucleotide sequence of or differing by 1 or 2 nucleotides from the nucleotide sequence set forth in SEQ ID NO.2 and a sense strand having a nucleotide sequence of or differing by 1 or 2 nucleotides from the nucleotide sequence set forth in SEQ ID NO.1;
2) an antisense strand having a nucleotide sequence of or differing by 1 or 2 nucleotides from the nucleotide sequence set forth in SEQ ID NO.4 and a sense strand having a nucleotide sequence of or differing by 1 or 2 nucleotides from the nucleotide sequence set forth in SEQ ID NO.3;
3) an antisense strand having a nucleotide sequence of or differing by 1 or 2 nucleotides from the nucleotide sequence set forth in SEQ ID NO.6 and a sense strand having a nucleotide sequence of or differing by 1 or 2 nucleotides from the nucleotide sequence set forth in SEQ ID NO.5;
4) an antisense strand having a nucleotide sequence of or differing by 1 or 2 nucleotides from the nucleotide sequence set forth in SEQ ID NO.8 and a sense strand having a nucleotide sequence of or differing by 1 or 2 nucleotides from the nucleotide sequence set forth in SEQ ID NO.7;
5) an antisense strand having a nucleotide sequence of or differing by 1 or 2 nucleotides from the nucleotide sequence set forth in SEQ ID NO.10 and a sense strand having a nucleotide sequence of or differing by 1 or 2 nucleotides from the nucleotide sequence set forth in SEQ ID NO.9;
6) an antisense strand having a nucleotide sequence of or differing by 1 or 2 nucleotides from the nucleotide sequence set forth in SEQ ID NO.12 and a sense strand having a nucleotide sequence of or differing by 1 or 2 nucleotides from the nucleotide sequence set forth in SEQ ID NO.11; and
7) an antisense strand having a nucleotide sequence of or differing by 1 or 2 nucleotides from the nucleotide sequence set forth in SEQ ID NO.13 and a sense strand having a nucleotide sequence of or differing by 1 or 2 nucleotides from the nucleotide sequence set forth in SEQ ID NO.14.

6. The compound according to claim 1, wherein each nucleotide in the double-stranded oligonucleotide is independently selected from the group consisting of:
2′-fluoro modified nucleotide, 2′-deoxy modified nucleotide, 2′-O-methyl modified nucleotide, 2′-O—(CH₂)ₓ—O—Rₘ modified nucleotide, 2′-O—Si(Rₐ)₃ modified nucleotide, 2′-amino-modified nucleotide, abasic nucleotide, and a nucleotide analogue, wherein the nucleotide analogue is one or more selected from the group consisting of PNA, MNA, BNA, LNA, GNA, TNA and UNA; wherein, x is selected from the group consisting of 1 and 2, Rₘ is selected from the group consisting of optionally substituted C₁₋₆ alkyl and optionally substituted C₁₋₆ alkoxy, when Rₘ comprises a substituent, the substituent is selected from the group consisting of halogen, C₁₋₃ alkyl and C₁₋₃ alkoxy; Rₙ is independently selected from unsubstituted or optionally substituted C₁₋₆ alkyl, when Rₙ comprises a substituent, the substituent is selected from the group consisting of halogen, C₁₋₃ alkyl and C₁₋₃ alkoxy;
optionally, the 2′-O—(CH₂)ₓ—O—Rₘ modified nucleotide is selected from the group consisting of a 2′-O-methoxyethyl-modified nucleotide and a 2′-O-ethoxymethyl-modified nucleotide;
optionally, the 2′-O—Si(Rₙ)₃ modified nucleotide is selected from the group consisting of a 2′-O-TBDMS modified nucleotide, a 2′-O-TIPS modified nucleotide and a 2′-O-TOM modified nucleotide; and
optionally, the double-stranded oligonucleotide comprises at least one 2′-O-methoxyethyl modified nucleotide.

7. The compound according to claim 1, wherein the double-stranded oligonucleotide is selected from the group consisting of:
in the direction from 5′ end to 3′ end, at least three nucleotides at positions 7 to 10 of the nucleotide sequence of the sense strand of the double-stranded oligonucleotide are 2′-fluoro modified nucleotides, and nucleotides at the other positions in the sense strand are other than 2′-fluoro modified nucleotides; nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, and nucleotides at the other positions in the antisense strand are other than 2′-fluoro modified nucleotides;
optionally, in the direction from 5′ end to 3′ end, at least three nucleotides at positions 7 to 10 of the nucleotide sequence of the sense strand of the double-stranded oligonucleotide are 2′-fluoro modified nucleotides, and nucleotides at the other positions in the sense strand are 2′-O-methyl modified nucleotides or 2′-O-methoxyethyl modified nucleotides;
nucleotides at positions 2, 6, 14, and 16 of the nucleotide sequence of the antisense strand are 2′-fluoro modified nucleotides, any one of nucleotides at positions 9, 10, 11, 12 is a 2′-fluoro modified nucleotide, and nucleotides at the other positions in the antisense strand are 2′-O-methyl modified nucleotides or 2′-O-methoxyethyl modified nucleotides;
optionally, in the direction from 5′ end to 3′ end, at least three nucleotides at positions 7 to 10 of the nucleotide sequence of the sense strand of the double-stranded oligonucleotide are 2′-fluoro modified nucleotides, at most two nucleotides at positions 5, 12, 18 are 2′-O-methoxyethyl modified nucleotides, and nucleotides at the other positions in the sense strand are 2′-O-methyl modified nucleotides; nucleotides at positions 2, 6, 14, and 16 of the nucleotide sequence of the antisense strand are 2′-fluoro modified nucleotides, any one of nucleotides at positions 9 to 12 is a 2′-fluoro modified nucleotide, a nucleotide at position 15 is a 2′-O-methoxyethyl-modified nucleotide, and nucleotides at the other positions in the antisense strand are 2′-O-methyl modified nucleotides;
optionally, in the direction from 5′ end to 3′ end, at least three nucleotides at positions 7 to 10 of the nucleotide sequence of the sense strand of the double-stranded oligonucleotide are 2′-fluoro modified nucleotides, and nucleotides at the other positions in the sense strand are 2′-O-methyl modified nucleotides; nucleotides at positions 2, 6, 14, and 16 of the nucleotide sequence of the antisense strand are 2′-fluoro modified nucleotides, any one of nucleotides at positions 9 to 12 is a 2′-fluoro modified nucleotide, a nucleotide at position 15 is a 2′-O-methoxyethyl-modified nucleotide, and nucleotides at the other positions in the antisense strand are 2′-O-methyl modified nucleotides;
optionally, in the direction from 5′ end to 3′ end, nucleotides at positions 7 to 10 of the nucleotide sequence of the sense strand of the double-stranded oligonucleotide are 2′-fluoro modified nucleotides, and nucleotides at the other positions in the sense strand are 2′-O-methyl modified nucleotides; nucleotides at positions 2, 6, 14, and 16 of the nucleotide sequence of the antisense strand are 2′-fluoro modified nucleotides, any one of nucleotides at positions 9 to 12 is a 2′-fluoro modified nucleotide, a nucleotide at position 15 is a 2′-O-methoxyethyl-modified nucleotide, and nucleotides at the other positions in the antisense strand are 2′-O-methyl modified nucleotides;
optionally, in the direction from the 5′ end to 3′ end, at least one of linkages between the following nucleotides of the sense strand is a phosphorothioate linkage: a linkage between the first nucleotide and the second nucleotide at 5′ end of the sense strand, and a linkage between the second nucleotide and the third nucleotide at 5′ end of the sense strand; and
optionally, in the direction from the 5′ end to 3′ end, at least one of linkages between the following nucleotides of the antisense strand is a phosphorothioate linkage: a linkage between the first nucleotide and the second nucleotide at 5′ end of the antisense strand, a linkage between the second nucleotide and the third nucleotide at 5′ end of the antisense strand, a linkage between the first nucleotide and the second nucleotide at 3′ end of the antisense strand, and a linkage between the second nucleotide and the third nucleotide at 3′ end of the antisense strand.

8. The compound according to claim 1, wherein each nucleotide in the double-stranded oligonucleotide is a modified nucleotide,
optionally, the double-stranded oligonucleotide is one or more sets selected from the group consisting of set 1, set 2, set 3, set 4, set 5, set 6, set 7 and set 8,

| | sense strand (5′-3′) | antisense strand(5′-3′) |
|---|---|---|
| set 1 | GmsGmsCmUmUmGmAfUfCfAf | AmsGfsUmAmGmUfUmCmAmUm |
| | UmGmAmAmCmUmAmCmUm | GfAmUmCfAmAfGmCmCmsAmsG |
| | | m |
| set 2 | GmsGmsCmUmUmGmAfUfCfAf | AmsGfsUmAmGmUfUmCmAmUm |
| | UmGmAmAmCmUmAmCmUm | GfAmUmCfA(moe)AfGmCmCm |
| | | sAmsGm |
| set 3 | GmsCmsAmGmCmUmCfCfUfUf | UmsAfsUmAmAmCfAmAmUmAm |
| | AmUmUmGmUmUmAmUmAm | AfGmGmAfGmCfUmGmCmsCmsA |
| | | m |
| set 4 | GmsCmsAmGmCmUmCfCfUfUf | UmsAfsUmAmAmCfAmAmUmAm |
| | AmUmUmGmUmUmAmUmAm | AfGmGmAfG(moe)CfUmGmCm |
| | | sCmsAm |
| set 5 | GmsCmsUmCmCmUmUfAfUfUf | UmsCfsGmUmAmUfAmAmCmAm |
| | GmUmUmAmUmAmCmGmAm | AfUmAmAfGmGfAmGmCmsUmsG |
| | | m |
| set 6 | CmsUmsGmGmCmUmUfGfAfUf | UmsAfsGmUmUmCfUmUmGmAm |
| | CmAmAmGmAmAmCmUmAm | UfCmAmAfGmCfCmAmGmsCmsA |
| | | m |
| set 7 | CmsUmsGmGmCmUmUfGfAfUf | UmsAfsGmUmUmCfUmUmGmAmU |
| | CmAmAmGmAmAmCmUmAm | fCmAmAfG(moe)CfCmAmGmsC |
| | | msAm |
| set 8 | CmsUmsGmAmCmAmCfAfAfUf | UmsUfsUmCmUmGfAmGmCmAm |
| | GmCmUmCmAmGmAmAmAm | UfUmGmUfG(moe)UfCmAmGms |
| | | AmsUm |

9. The compound according to claim 1, wherein the compound is any one selected from the compounds shown in Table 6;
optionally, the compound is any one selected from the group consisting of RZOO1031, RZ001032, RZ001033, RZ001034, RZ001035, RZ001038, RZ001039, RZ001044:

| | sense strand (5′-3′) | antisense strand (5′-3′) |
|---|---|---|
| RZ001031 | GmsGmsCmUmUmGmAfUfCfA | AmsGfsUmAmGmUfUmCmAmUm |
| | fUmGmAmAmCmUmAmCmUm | GfAmUmCfAmAfGmCmCmsAmsG |
| | _(CR01008×3) | m |
| RZ001032 | GmsGmsCmUmUmGmAfUfCfA | AmsGfsUmAmGmUfUmCmAmUm |
| | fUmGmAmAmCmUmAmCmUm | GfAmUmCfA(moe)AfGmCmCmsA |
| | _(CR01008×3) | msGm |
| RZ001033 | GmsCmsAmGmCmUmCfCfUfUf | UmsAfsUmAmAmCfAmAmUmAm |
| | AmUmUmGmUmUmAmUmAm | AfGmGmAfGmCfUmGmCmsCmsA |
| | _(CR01008×3) | m |
| RZ001034 | GmsCmsAmGmCmUmCfCfUfUf | UmsAfsUmAmAmCfAmAmUmAm |
| | AmUmUmGmUmUmAmUmAm | AfGmGmAfG(moe)CfUmGmCmsC |
| | _(CR01008×3) | msAm |
| RZ001035 | GmsCmsUmCmCmUmUfAfUfUf | UmsCfsGmUmAmUfAmAmCmAm |
| | GmUmUmAmUmAmCmGmAm | AfUmAmAfGmGfAmGmCmsUms |
| | _(CR01008×3) | Gm |
| RZ001038 | CmsUmsGmGmCmUmUfGfAfU | UmsAfsGmUmUmCfUmUmGmAm |
| | fCmAmAmGmAmAmCmUmAm | UfCmAmAfGmCfCmAmGmsCmsA |
| | _(CR01008×3) | m |
| RZ001039 | CmsUmsGmGmCmUmUfGfAfU | UmsAfsGmUmUmCfUmUmGmAm |
| | fCmAmAmGmAmAmCmUmAm | UfCmAmAfG(moe)CfCmAmGmsC |
| | _(CR01008×3) | msAm |
| RZ001044 | CmsUmsGmAmCmAmCfAfAfUf | UmsUfsUmCmUmGfAmGmCmAm |
| | GmCmUmCmAmGmAmAmAm | UfUmGmUfG(moe)UfCmAmGmsA |
| | _(CR01008×3) | msUm |

optionally, the compound is RZ001032.

10. A pharmaceutical composition comprising the compound according to claim 1 and a pharmaceutically acceptable excipient.

11. (canceled)

12. A kit comprising the compound according to claim 1.

13. A method for inhibiting expression of LPA gene in a subject in need thereof, comprising administering to the subject the compound according to claim 1.

14. A method for alleviating, treating and/or preventing a LPA-mediated disease or condition in a subject in need thereof, comprising administering to the subject the compound according to claim 1,
optionally, the LPA gene-mediated disease or condition includes a disease related to mRNA expression level of LPA gene, and
optionally, the LPA gene-mediated disease or condition includes cardiovascular disease, the cardiovascular disease includes but is not limited to hyperlipoproteinemia(a), Berger's disease, peripheral artery disease, coronary artery disease, metabolic syndrome, acute coronary syndrome, aortic stenosis, aortic regurgitation, aortic dissection, cerebrovascular disease, mesenteric ischemia, superior mesenteric artery occlusion, renal artery stenosis, stable/unstable angina pectoris, acute coronary syndrome, heterozygous or homozygous familial hypercholesterolemia, high apolipoprotein beta lipoprotein, cerebrovascular atherosclerosis, cerebrovascular disease and venous thrombosis, stroke, atherosclerosis, thrombosis, coronary artery disease and/or any other disease or condition related to the increased level of LP(a) particles.

15. A kit comprising the pharmaceutical composition according to claim 10.

16. A method for inhibiting expression of LPA gene in a subject in need thereof, comprising administering to the subject the pharmaceutical composition according to claim 10.

17. A method for alleviating, treating and/or preventing a LPA-mediated disease or condition in a subject in need thereof, comprising administering to the subject the pharmaceutical composition according to claim 10,
optionally, the LPA gene-mediated disease or condition includes a disease related to mRNA expression level of LPA gene, and
optionally, the LPA gene-mediated disease or condition includes cardiovascular disease, the cardiovascular disease includes but is not limited to hyperlipoproteinemia(a), Berger's disease, peripheral artery disease, coronary artery disease, metabolic syndrome, acute coronary syndrome, aortic stenosis, aortic regurgitation, aortic dissection, cerebrovascular disease, mesenteric ischemia, superior mesenteric artery occlusion, renal artery stenosis, stable/unstable angina pectoris, acute coronary syndrome, heterozygous or homozygous familial hypercholesterolemia, high apolipoprotein beta lipoprotein, cerebrovascular atherosclerosis, cerebrovascular disease and venous thrombosis, stroke, atherosclerosis, thrombosis, coronary artery disease and/or any other disease or condition related to the increased level of LP(a) particles.
